SEQUENCE LISTING (8 sequences)

SEQ ID NO: 1 - DNA, 20 bp, synthetic construct
  source 1..20: mol_type = other DNA
ctggatatcaacctcagcgg

SEQ ID NO: 2 - DNA, 20 bp, synthetic construct
  source 1..20: mol_type = other DNA
gattcaggtcgaggttcagc

SEQ ID NO: 3 - DNA, 20 bp, synthetic construct
  source 1..20: mol_type = other DNA
gggtaacgccagggttttcc

SEQ ID NO: 4 - DNA, 20 bp, synthetic construct
  source 1..20: mol_type = other DNA
ctggatatcaacctcagcgg

SEQ ID NO: 5 - DNA, 19 bp, synthetic construct
  source 1..19: mol_type = other DNA
gtcgcggatatcgatcagg

SEQ ID NO: 6 - DNA, 20 bp, synthetic construct
  source 1..20: mol_type = other DNA
aagggcatctacattctcgc

SEQ ID NO: 7 - DNA, 20 bp, synthetic construct
  source 1..20: mol_type = other DNA
gggtaacgccagggttttcc

SEQ ID NO: 8 - DNA, 20 bp, synthetic construct
  source 1..20: mol_type = other DNA
aagggcatctacattctcgc

(19) United States
(12) Patent Application Publication
(10) Pub. No.: US 2026/0224658 A1
(43) Pub. Date: Aug. 6, 2026

(54) PRODUCTS AND METHODS FOR THE DIAGNOSIS AND TREATMENT OF PSEUDOMONAS INFECTIONS

(71) Applicant: The Administrators of the Tulane Educational Fund, New Orleans, LA (US)

(72) Inventors: Kalen M. HALL, New Orleans, LA (US); Leonard G. WILLIAMS, New Orleans, LA (US); Lisa A. MORICI, New Orleans, LA (US); Zac PURSELL, New Orleans, LA (US)

(73) Assignee: The Administrators of the Tulane Educational Fund, New Orleans, LA (US)

(21) Appl. No.: 19/153,250

(22) PCT Filed: Feb. 2, 2024
(86) PCT No.: PCT/US2024/014280

§ 371 Date: Aug. 1, 2025

Related U.S. Application Data
(60) Provisional application No. 63/443,340, filed on Feb. 3, 2023.

(51) Int. Cl. A61K 38/12; A61K 31/426; A61K 38/10; A61P 31/04; C12Q 1/689

(52) U.S. Cl. CPC A61K 38/12; A61K 31/426; A61K 38/10; A61P 31/04; C12Q 1/689; C12Q 2600/106; C12Q 2600/118; G01N 2800/26

(57) ABSTRACT

The present disclosure provides methods of diagnosing and treating Pseudomonas infections in a subject in need thereof based on the mutational signature (e.g., DNA mismatch repair-deficit (dMMR) mutational signature) of the Pseudomonas bacteria. These methods are based on detecting DNA mismatch repair-deficit (dMMR) mutational signature of Pseudomonas species.

### CROSS-REFERENCE TO RELATED APPLICATIONS

This application claims the benefit of and priority to U.S. Provisional Patent Application No. 63/443,340, filed Feb. 3, 2023, the contents of which are incorporated herein by reference in its entirety.

### GOVERNMENT SUPPORT

This invention was made with government support under 5R21AI154284-02 under National Institutes of Health. The government has certain rights in the invention.

### TECHNICAL FIELD

The present disclosure provides methods of diagnosis and treatment of Pseudomonas infections in a subject in need thereof based on the mutational signature (e.g., DNA mismatch repair-deficit (dMMR) mutational signature) of the Pseudomonas bacteria. In one aspect, the present disclosure provides methods of diagnosis and treatment of hypermutator Pseudomonas aeruginosa infection based on the dMMR Pseudomonas aeruginosa mutational signature.

### BACKGROUND

The following description of the background of the present technology is provided simply as an aid in understanding the present technology and is not admitted to describe or constitute prior art to the present technology.

Hypermutation appears to be an evolutionary conserved mechanism for cells to rapidly adapt to everchanging selective pressures and has been observed across human cancer cells to clinical bacterial isolates. In bacteria, hypermutator alleles commonly become fixed in a population due to the evolutionary advantages they confer. Hypermutators can rapidly adapt to stringent selective pressures, such as antibiotics and host immune responses, and hypermutator alleles ‘hitchhike’ along with these downstream mutations.

Hypermutators comprise up to 60% of chronic respiratory Pseudomonas aeruginosa isolates and have been documented in isolates from cystic fibrosis (CF) patients. Hypermutator isolates have been strongly linked to multidrug resistance and treatment failure in CF patients.

Accordingly, there is an urgent need for effective methods for diagnosing and treating antibiotic-resistant bacterial infections, such as hypermutator Pa.

### SUMMARY OF THE PRESENT TECHNOLOGY

In one aspect, the present disclosure provides a method for treating a hypermutator Pseudomonas infection in a subject in need thereof comprising administering to the subject an effective amount of an antipseudomonal small molecule inhibitor and an effective amount of an antipseudomonal cationic peptide. In another aspect, the present disclosure provides a method for preventing hypermutator Pseudomonas-induced antibiotic resistance in a subject having a hypermutator Pseudomonas infection comprising administering to the subject an effective amount of an antipseudomonal small molecule inhibitor and an effective amount of an antipseudomonal cationic peptide. In some embodiments, the antipseudomonal cationic peptide comprises one or more of polymyxin B, D-CONGA, D-CONGA-Q7 or colistin. Additionally or alternatively, in certain embodiments, the antipseudomonal small molecule inhibitor comprises one or more of monobactams, fluoroquinolines, cephalosporins, or carbapenems. Examples of monobactams include, but are not limited to, aztreonam, azactam, tigemonam, nocardicin A, tabtoxin, and cayston. Examples of fluoroquinolines include, but are not limited to, ciprofloxacin, gemifloxacin, levofloxacin, delafloxacin, gemifloxacin, moxifloxacin, norfloxacin, and ofloxacin. Examples of cephalosporins include, but are not limited to, cephalexin, cefadroxil, cephradine, cephalexin, cefazolin, cefuroxime, cefprozil, loracarbef, cefuroxime, cefoxitin, cefotetan, ceftriaxone, cefdinir, cefixime, cefpodoxime, cefditoren, ceftibuten, cefdinir, ceftazidime, cefotaxime, cefoperazone, ceftizoxime, cefepime, ceftaroline, ceftolozane, or cefiderocol. Examples of carbapenems include, but are not limited to, doribax, doripenem, ertapenem, imipenem, cilastatin, relebactam, invanz, meropenem, vaborbactam, merrem IV, primaxin, recarbrio, sulopenem, sulopenem etzadroxil/probenecid, or vabomere.

In any of the preceding embodiments of the methods disclosed herein, the Pseudomonas infection is caused by a Pseudomonas species selected from among P. aeruginosa, P. fluorescens, P. putida, P. cepacia, P. stutzeri, P. maltophilia, and P. putrefaciens. In certain embodiments, the Pseudomonas infection is caused by P. aeruginosa. Additionally or alternatively, in some embodiments, the subject is diagnosed with or is at risk for cystic fibrosis or an acute infection. The acute infection may comprise a pressure sore infection, a burn infection, a wound infection or bloodstream infection.

In any and all embodiments of the methods disclosed herein, the antipseudomonal small molecule inhibitor and the antipseudomonal cationic peptide is administered separately, sequentially, or simultaneously. Additionally or alternatively, in some embodiments, the antipseudomonal small molecule inhibitor or the antipseudomonal cationic peptide is administered orally, intravenously, intramuscularly, intraperitoneally, or subcutaneously.

In one aspect, the present disclosure provides a method for detecting a hypermutator Pseudomonas infection in a subject comprising (a) sequencing Pseudomonas nucleic acids isolated from a biological sample obtained from the subject; (b) generating a mutation spectrum of the Pseudomonas nucleic acid sequences; and (c) detecting the presence of a hypermutator Pseudomonas infection when a Pseudomonas DNA mismatch repair-deficit (dMMR) mutational signature is detected in the mutation spectrum. In another aspect, the present disclosure provides a method for predicting the risk of antibiotic resistance in a subject having a Pseudomonas infection comprising (a) sequencing Pseudomonas nucleic acids isolated from a biological sample obtained from the subject; (b) generating a mutation spectrum of the Pseudomonas nucleic acid sequences; and (c) determining that the subject is at risk for antibiotic resistance when a Pseudomonas DNA mismatch repair-deficit (dMMR) mutational signature is detected in the mutation spectrum. The Pseudomonas nucleic acid sequences may comprise genomic DNA or cell-free DNA. Additionally or alternatively, in certain embodiments, the dMMR mutational signature comprises an increase in C>T transitions in NCC and NCG contexts and/or an increase in T>C transitions in CTN and GTN contexts. In some embodiments, the dMMR mutational signature comprises an increase in C>T transitions in ACC, GCC, TCC, CCC, ACG, GCG, TCG, and/or CCG contexts. Additionally or alternatively, in certain embodiments, the dMMR mutational signature comprises an increase in T>C transitions in CTA, CTG, CTC, CTT, GTA, GTC, GTG, and/or GTT. The biological sample may comprise skin tissue, throat swabs, stool, urine, blood, lung tissue, stomach tissue, or urinary tract tissue. In certain embodiments, the methods of the present technology further comprise administering to the subject an effective amount of an antipseudomonal small molecule inhibitor and an effective amount of an antipseudomonal cationic peptide.

In one aspect, the present disclosure provides a method for preventing multi-drug resistance in a subject infected with Pseudomonas comprising (a) sequencing Pseudomonas nucleic acids isolated from a biological sample obtained from the subject; (b) generating a mutation spectrum of the Pseudomonas nucleic acid sequences; (c) detecting a Pseudomonas DNA mismatch repair-deficit (dMMR) mutational signature in the mutation spectrum; and (d) administering to the subject an effective amount of an antipseudomonal small molecule inhibitor and an effective amount of an antipseudomonal cationic peptide. In another aspect, the present disclosure provides a method for selecting an antipseudomonal therapy for a subject having a Pseudomonas infection comprising (a) sequencing Pseudomonas nucleic acids isolated from a biological sample obtained from the subject; (b) generating a mutation spectrum of the Pseudomonas nucleic acid sequences; (c) detecting a Pseudomonas DNA mismatch repair-deficit (dMMR) mutational signature in the mutation spectrum; and (d) administering to the subject an effective amount of an antipseudomonal small molecule inhibitor and an effective amount of an antipseudomonal cationic peptide. The Pseudomonas nucleic acid sequences may comprise genomic DNA or cell-free DNA. Additionally or alternatively, in certain embodiments, the dMMR mutational signature comprises an increase in C>T transitions in NCC and NCG contexts and/or an increase in T>C transitions in CTN and GTN contexts. In some embodiments, the dMMR mutational signature comprises an increase in C>T transitions in ACC, GCC, TCC, CCC, ACG, GCG, TCG, and/or CCG contexts. Additionally or alternatively, in certain embodiments, the dMMR mutational signature comprises an increase in T>C transitions in CTA, CTG, CTC, CTT, GTA, GTC, GTG, and/or GTT. In some embodiments, the Pseudomonas DNA mismatch repair-deficit (dMMR) mutational signature is detected by using a cosine similarity of the comparison between the mutation spectrum of Pseudomonas nucleic acid sequences obtained from the biological sample with a mutation spectrum of nucleic acid sequences obtained from a control hypermutator Pseudomonas reference strain.

In any of the preceding embodiments of the methods disclosed herein, the Pseudomonas infection is caused by a Pseudomonas species selected from among P. aeruginosa, P. fluorescens, P. putida, P. cepacia, P. stutzeri, P. maltophilia, and P. putrefaciens. In some embodiments, the antipseudomonal cationic peptide comprises one or more of polymyxin B, D-CONGA, D-CONGA-Q7 or colistin. Additionally or alternatively, in certain embodiments, the antipseudomonal small molecule inhibitor comprises one or more of monobactams, fluoroquinolines, cephalosporins, or carbapenems. Examples of monobactams include, but are not limited to, aztreonam, azactam, tigemonam, nocardicin A, tabtoxin, and cayston. Examples of fluoroquinolines include, but are not limited to, ciprofloxacin, gemifloxacin, levofloxacin, delafloxacin, gemifloxacin, moxifloxacin, norfloxacin, and ofloxacin. Examples of cephalosporins include, but are not limited to, cephalexin, cefadroxil, cephradine, cephalexin, cefazolin, cefuroxime, cefprozil, loracarbef, cefuroxime, cefoxitin, cefotetan, ceftriaxone, cefdinir, cefixime, cefpodoxime, cefditoren, ceftibuten, cefdinir, ceftazidime, cefotaxime, cefoperazone, ceftizoxime, cefepime, ceftaroline, ceftolozane, or cefiderocol. Examples of carbapenems include, but are not limited to, doribax, doripenem, ertapenem, imipenem, cilastatin, relebactam, invanz, meropenem, vaborbactam, merrem IV, primaxin, recarbrio, sulopenem, sulopenem etzadroxil/probenecid, or vabomere.

In any of the preceding embodiments of the methods disclosed herein, Pseudomonas nucleic acids are sequenced via whole genome sequencing (WGS). In any and all embodiments of the methods disclosed herein, the mutation spectrum comprises a trinucleotide mutation spectrum of SNVs present in the Pseudomonas nucleic acid sequences.

### BRIEF DESCRIPTION OF THE DRAWINGS

FIGS. 1A-1D: MMR-deficient MPAO1-ΔmutS possesses a hallmark mutational signature of MMR-deficiency and catalyzes drug resistance acquisition. FIG. 1A: The mutation rate of MPAO1-ΔmutS evolved lineages compared to MPAO1 (WT) is shown. FIG. 1B: A compilation of mutation spectra of identified unique SNVs in a trinucleotide context SNVs from 27 independent MPAO1-ΔmutS samples is shown. FIG. 1C: Aztreonam resistance acquisition of MPAO1-ΔmutS compared to MPAO1 is shown. FIG. 1D: Colistin (COL), D-CONGA, and D-CONGA-Q7 resistance acquisition of MPAO1-ΔmutS compared to MPAO1 is shown. n: number. *p<0.05; **p<0.01, ***p<0.001, and ****p<0.0001. One-way ANOVA with multiple comparisons with Bonferroni correction of slopes from linear regression analysis of log-transformed datasets was used.

FIGS. 2A-2D: Treatment of MMR-deficient MPAO1-ΔmutS selectively induces multidrug resistance through shared mechanisms of resistance. FIG. 2A: Ciprofloxacin cross-resistance acquisition of MPAO1-ΔmutS compared to MPAO1 is shown. The black dashed line represents the clinical breakpoint designated by CLSI. FIG. 2B: D-CONGA and D-CONGA-Q7 cross-resistance acquisition of MPAO1-ΔmutS compared to MPAO1 is shown.

FIG. 2C: MALDI-MS data from lipid A isolated from 3 independent MPAO1-ΔmutS lineages evolved in D-CONGA-Q7 (top) or D-CONGA (bottom) compared to MPAO1 (top right) is shown. FIG. 2D: A mutation heatmap showing parallel evolution of nonsynonymous mutations in efflux pumps and membrane-modifying genes for non-peptide and peptide antibiotic treated lineages, respectively, is shown. AZ: aztreonam, Chl: chloramphenicol, COL: colistin. *p<0.05; **p<0.01, ***p<0.001, and ****p<0.0001. One-way ANOVA of slopes from linear regression analysis of log-transformed datasets was used.

FIGS. 3A-3D: Rational combination therapy prevents multidrug resistance acquisition in MMR-deficient MPAO1-ΔmutS. FIG. 3A: Resistance acquisition of MPAO1-ΔmutS exposed to aztreonam+colistin, aztreonam only, or colistin only is shown. FIG. 3B: Aztreonam cycled with tobramycin resistance acquisition of MPAO1-ΔmutS compared to MPAO1 is shown. FIG. 3C: Polymyxin B cross-resistance acquisition of MPAO1-ΔmutS exposed to combination therapy is shown. FIG. 3D: Ciprofloxacin cross-resistance acquisition of MPAO1-ΔmutS exposed to combination therapy is shown. AB: antibiotic, AZ: aztreonam, TOB: tobramycin, COL: colistin. *p<0.05; **p<0.01, ***p<0.001, and ****p<0.0001. One-way ANOVA of slopes (FIG. 3A), unpaired t-test (FIG. 3B), or one-way ANOVA with multiple comparisons (FIGS. 3C-3D) from linear regression analysis of log-transformed datasets was used.

FIGS. 4A-4H Mutational signature analysis predicts MMR-deficient clinical isolates which are susceptible to combination, but not monotherapy. FIG. 4A: Representative trinucleotide mutation spectra of predicted MMR-deficient isolates (top) and predicted WT isolates (bottom) is shown. FIG. 4B: Unique SNVs and indels per isolate following deduplication of variants across all samples, with predicted MMR-deficient isolates colored is shown in light gray font. FIG. 4C: A cosine similarity heatmap of trinucleotide mutation spectra from patient isolates compared to the P. aeruginosa MMR-deficiency mutational signature (Pa ΔmutS), composite human signature (Human ΔMMR), and each individual COSMIC SBS associated with MMR-deficiency is shown. Samples are clustered based on cosine similarity. FIG. 4D: Mutant frequency via rifampicin reversion (rpoB mutants resistant to rifampicin per 10⁸ viable cells) of all patient isolates functionally validated predictions of MMR-status is shown. Hypermutators are defined as 20-fold higher than WT parent strain (dotted line). Lab strains are colored as white, predicted WT clinical isolates as grey, and predicted MMR-deficient clinical isolates as dark grey. FIG. 4E: Numbers of nonsynonymous mutations in key drug efflux or membrane-modifying genes in all patient isolates is shown. Heat map is stratified by predicted MMR-deficient isolates (top 7 isolates) and predicted WT isolates (bottom 19 isolates). FIG. 4F: Aztreonam resistance acquisition of eight representative patient P. aeruginosa isolates is shown. FIG. 4G: Colistin resistance acquisition of eight representative patient P. aeruginosa isolates is shown. FIG. 4H: Resistance acquisition of predicted MMR-deficient isolate CFP6_Pa2 exposed to aztreonam+colistin, aztreonam only, or colistin only is shown. n: number, AZ: aztreonam, COL: colistin. *p<0.05; **p<0.01, ***p<0.001, and ****p<0.0001. One-way ANOVA of slopes from linear regression analysis of log-transformed datasets was used.

FIGS. 5A-5F. Mutational signature analysis predicts MMR-deficiency and multi-drug resistance (MDR) in P. aeruginosa isolates from pwCF. FIG. 5A: Representative trinucleotide mutation spectra of patient isolates predicted as MMR-deficient is shown. FIG. 5B: Cosine similarity analysis of trinucleotide mutation spectra from patient isolates compared to the P. aeruginosa MMR-deficiency mutational signature (Pa ΔmutS), composite human signature (Human ΔMMR), and each individual COSMIC SBS associated with MMR-deficiency is shown. Samples are clustered based on cosine similarity. FIG. 5C: Proportion of predicted MMR-deficient and WT isolates containing nonsynonymous mutation in MMR gene(s) is shown. FIG. 5D: Comparison of predictions using mutational signature analysis versus mutator phenotype reported by Lopez-Causape et al. is shown. FIGS. 5D-5F: Correlation of predicted MMR-deficiency using mutational signature analysis (FIG. 5E) or hypermutation from rifampicin reversion (FIG. 5F) with MDR. p=0.0021 (FIG. 5E) and 0.0009 (FIG. 5F), Fisher's exact test is shown. MDR isolates were defined by clinical resistance (‘R’ via EUCAST) to ≥3 drugs. n: number.

FIGS. 6A-6G. Mutational signature analysis reveals presence of MMR-deficiency in other contexts. FIG. 6A. Trinucleotide mutation spectra of all patient isolates predicted as MMR-deficient, with n=number, CF=cystic fibrosis, RTI=respiratory tract infection, UTI=urinary tract infection, IAI=intraabdominal infection is shown. FIG. 6B: Cosine similarity analysis of trinucleotide mutation spectra from patient isolates compared to the P. aeruginosa MMR-deficiency mutational signature (Pa ΔmutS), composite human signature (Human ΔMMR), and each individual COSMIC SBS associated with MMR-deficiency is shown. Samples are clustered based on cosine similarity. FIG. 6C: Proportion of predicted MMR-deficiency across disease contexts is shown. Bars represent means±95% CI, via Wilson/Brown. FIGS. 6D-6G: Proportion of MDR isolates across predicted MMR-deficient versus predicted WT isolates in CF (FIG. 6D), RTI (FIG. 6E), UTI (FIG. 6F), and IAI (FIG. 6G) is shown. MDR is defined by clinical resistance (‘R’ via CLSI) to ≥2 drugs. p=0.0256 (FIG. 6D) via Fisher's exact.

FIGS. 7A-7B: Mutant frequency and characterization of SNVs. FIG. 7A: The mutant frequency via rifampicin reversion (rpoB mutants resistant to rifampicin per 10⁸ viable cells) of MPAO1-ΔmutS compared to MPAO1 is shown. Hypermutators are defined as 20-fold higher than wild-type or parent strain (black dotted line). FIG. 7B: The proportion SNVs containing transitions and indels in homopolymers for unique SNVs of MPAO1-ΔmutS compared to MPAO1 (WT) is shown.

FIG. 8: The trinucleotide spectra of de novo SNVs of individual independently evolved clones of MPAO1-ΔmutS is shown. AB: antibiotic, AZ: aztreonam, Chl: chloramphenicol.

FIGS. 9A-9B. Resistance acquisition of MPAO1-ΔmutL. FIG. 9A: Aztreonam resistance acquisition of MPAO1-ΔmutL compared to MPAO1 is shown. The black dashed line represents clinical breakpoint designated by CLSI. FIG. 9B: Colistin resistance acquisition of MPAO1-ΔmutL compared to MPAO1 is shown. The black dashed line represents clinical breakpoint designated by CLSI. AZ: aztreonam, COL: colistin. *p<0.05; **p<0.01, ***p<0.001, and ****p<0.0001. One-way t-tests of slopes from linear regression analysis of log-transformed datasets was used.

FIGS. 10A-10B. Aztreonam cross-resistance acquisition of MPAO1-ΔmutS and MPAO1-ΔmutL. FIG. 10A: Aztreonam cross-resistance acquisition of MPAO1-ΔmutS compared to MPAO1 is shown. The black dashed line represents clinical breakpoint designated by CLSI. FIG. 10B: Aztreonam cross-resistance acquisition of MPAO1-ΔmutL compared to MPAO1 is shown. The black dashed line represents clinical breakpoint designated by CLSI. AB: antibiotic, AZ: aztreonam, COL: colistin, Chl: chloramphenicol, Str: streptomycin. *p<0.05; **p<0.01, ***p<0.001, and ****p<0.0001. One-way ANOVA of slopes from linear regression analysis of log-transformed datasets was used.

FIGS. 11A-11B. Polymyxin B cross-resistance acquisition of MPAO1-ΔmutS. FIG. 11A: Polymyxin B cross-resistance acquisition of MPAO1-ΔmutS, exposed to peptides, compared to MPAO1 is shown. The black dashed line represents clinical breakpoint designated by CLSI. FIG. 11B: Polymyxin B cross-resistance acquisition of MPAO1-ΔmutS, exposed to antibiotics, compared to MPAO1 is shown. The black dashed line represents clinical breakpoint designated by CLSI. AB: antibiotic, AZ: aztreonam, COL: colistin, Chl: chloramphenicol. *p<0.05; **p<0.01, ***p<0.001, and ****p<0.0001. One-way ANOVA of slopes from linear regression analysis of log-transformed datasets was used.

FIG. 12: The allelic frequency of variant wbpL (C)_{9→10} (221/1020 nt) in peptide exposed MPAO1-ΔmutS is shown. Only subclonal (less than 100% reads) variants were analyzed.

FIGS. 13A-13B. Resistance acquisition of MPAO1-ΔmutS and MPAO1-ΔmutL exposed to combination therapy. FIG. 13A: Resistance acquisition of MPAO1-ΔmutS exposed to aztreonam+D-CONGA, aztreonam only, or D-CONGA only is shown (top). Resistance acquisition of MPAO1-ΔmutS exposed to aztreonam+D-CONGA-Q7, aztreonam only, or D-CONGA-Q7 only is shown (bottom). FIG. 13B: Resistance acquisition of MPAO1-ΔmutL exposed to aztreonam+colistin, aztreonam only, or colistin only is shown. AZ: aztreonam, COL: colistin. *p<0.05; **p<0.01, ***p<0.001, and ****p<0.0001. One-way ANOVA of slopes from linear regression analysis of log-transformed datasets was used.

FIGS. 14A-14C. Resistance acquisition of MPAO1 exposed to combination therapy. FIG. 14A: Resistance acquisition of MPAO1 exposed to aztreonam+colistin, aztreonam only, or colistin only is shown. FIG. 14B: Resistance acquisition of MPAO1 exposed to aztreonam+D-CONGA, aztreonam only, or D-CONGA only is shown. FIG. 14C: Resistance acquisition of MPAO1 exposed to aztreonam+D-CONGA-Q7, aztreonam only, or D-CONGA-Q7 only is shown. AZ: aztreonam, COL: colistin

FIGS. 15A-15C. Mutation characteristics of MPAO1-ΔmutS exposed to combination therapy. FIG. 15A: The mutation rate of MPAO1-ΔmutS unexposed to antibiotic compared to aztreonam+D-CONGA or aztreonam+D-CONGA-Q7 exposed MPAO1-ΔmutS is shown. FIG. 15B: The proportion of total unique mutations of MPAO1-ΔmutS unexposed to antibiotic compared to aztreonam+D-CONGA or aztreonam+D-CONGA-Q7 exposed MPAO1-ΔmutS is shown. FIG. 15C: The trinucleotide spectra of de novo SNVs of individual independently evolved clones of aztreonam+D-CONGA (top) or aztreonam+D-CONGA-Q7 (bottom) exposed MPAO1-ΔmutS is shown. n: number.

FIGS. 16A-16E: Resistance acquisition to monotherapies after repeated exposure to combination therapies. FIG. 16A: D-CONGA cross-resistance acquisition of MPAO1-ΔmutS is shown. FIG. 16B: D-CONGA-Q7 cross-resistance acquisition of MPAO1-ΔmutS is shown. FIG. 16C: Aztreonam or colistin resistance acquisition of MPAO1-ΔmutS following combo exposure with both is shown. FIG. 16D: Aztreonam or D-CONGA resistance acquisition of MPAO1-ΔmutS following combo exposure with both is shown. FIG. 16E: Aztreonam or D-CONGA-Q7 resistance acquisition of MPAO1-ΔmutS following combo exposure with both is shown. AB: antibiotic, AZ: aztreonam, COL: colistin. *p<0.05; **p<0.01, ***p<0.001, and ****p<0.0001. One-way ANOVA of slopes from linear regression analysis of log-transformed datasets was used.

FIG. 17: A compilation of all five MMR-deficiency associated COSMIC mutational signatures (SBS6, SBS15, SBS21, SBS26, SBS44) computationally extracted from human tumor genomes is shown. p: probability.

FIG. 18: The mutant frequency via rifampicin reversion (rpoB mutants resistant to rifampicin per 10⁸ viable cells) of all patient isolates is shown.

FIGS. 19A-19B: Resistance acquisition of three predicted MMR-deficient subject isolates exposed to combination therapy. FIG. 19A: Resistance acquisition of three predicted MMR-deficient subject isolates following exposure with aztreonam, colistin, or combo exposure with both is shown. FIG. 19B: Resistance acquisition of nine predicted wildtype subject isolates following exposure with aztreonam, colistin, or combo exposure with both is shown. AZ: aztreonam, COL: colistin. *p<0.05; **p<0.01, ***p<0.001, and ****p<0.0001. One-way ANOVA with multiple comparisons of slopes from linear regression analysis of log-transformed datasets was used.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

As described herein, drug treatment of MMR-deficient P. aeruginosa rapidly induces cross-resistance to non-treatment drugs, which is mediated by acquired resistance mechanisms (e.g., drug efflux) to the initial drug. These results also demonstrate that antibiotic treatment of MMR-deficient P. aeruginosa could rapidly drive pan-resistance, and empiric cycling of antibiotics in MMR-deficient P. aeruginosa infections could quickly reduce the number of effective treatment options for patients. As described in the Examples herein, antibiotic+cationic peptide combination therapy effectively prevents resistance acquisition in MMR-deficient P. aeruginosa compared to monotherapies or the standard of care therapies in vitro. For example, aztreonam+colistin treatment was even effective against MMR-deficient clinical isolates with preexisting aztreonam resistance. Without wishing to be bound by theory, addition of a drug imposing a distinct selective pressure prevents resistance acquisition to a different drug when applied in a rational combination. The present disclosure further demonstrates that MMR-deficient mutational signature is predictive of hypermutator status (and thus likely prone to MDR) and are thus suitable for antibiotic+cationic peptide combination therapy.

### Definitions

Unless defined otherwise, all technical and scientific terms used herein generally have the same meaning as commonly understood by one of ordinary skill in the art to which this technology belongs. As used in this specification and the appended claims, the singular forms “a” “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, a “control” is an alternative sample used in an experiment for comparison purpose. A control can be “positive” or “negative.” A “control nucleic acid sample” or “reference nucleic acid sample” as used herein, refers to nucleic acid molecules from a control or reference sample. In certain embodiments, the reference or control nucleic acid sample is a wild type or a non-mutated DNA or RNA sequence.

“Detecting” as used herein refers to determining the presence of a mutational signature in a sample comprising a Pseudomonas species. Detection does not require the method to provide 100% sensitivity. Analysis of nucleic acid markers can be performed using techniques known in the art including, but not limited to, sequence analysis, and electrophoretic analysis. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol, 3:39-42 (1992)), sequencing with mass spectrometry such as matrix-assisted laser desorption/ionization time-of-flight mass spectrometry (MALDI-TOF/MS; Fu et al., Nat. Biotechnol, 16:381-384 (1998)), and sequencing by hybridization. Chee et al., Science, 274:610-614 (1996); Drmanac et al., Science, 260:1649-1652 (1993); Drmanac et al., Nat. Biotechnol, 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

“Gene” as used herein refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

As used herein, “hypermutator” refers to a phenotype that exhibits rapid adaptive evolution and antibiotic resistance acquisition.

As used herein, the terms “individual”, “patient”, or “subject” are used interchangeably and refer to an individual organism, a vertebrate, a mammal, or a human. In a preferred embodiment, the individual, patient or subject is a human.

As used herein, a “mutation” of a gene refers to the presence of a variation within the gene or gene product that affects the expression and/or activity of the gene or gene product as compared to the normal or wild-type gene or gene product. The genetic mutation can result in changes in the quantity, structure, and/or activity of the gene or gene product in a mutant bacterial cell, as compared to its quantity, structure, and/or activity, in a control bacterial cell.

As used herein, a “mutational signature” is a discreet probability distribution of genetic mutations made by a specific mutagenic process. The specific mutagenic process may be one that occurs in nature, such as a mutagenic process in a bacterium that responds to a naturally occurring stressor in a native bacterial environment. In other embodiments, the specific mutagenic process may be intentionally induced, such as induction of a mutagenic process in a bacterium by treating the bacterium with an antibiotic.

“Next-generation sequencing or NGS” as used herein, refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 10³, 10⁴, 10⁵ or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. Nature Biotechnology Reviews 11:31-46 (2010).

As used herein, the term “sample” refers to clinical samples obtained from a patient or isolated microorganisms. In preferred embodiments, a sample is obtained from a biological source (i.e., a “biological sample”), such as tissue, bodily fluid, or microorganisms collected from a subject. Sample sources include, but are not limited to, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material).

The term “sensitivity,” as used herein in reference to the methods of the present technology, is a measure of the ability of a method to detect a preselected sequence variant in a heterogeneous population of sequences. A method has a sensitivity of S % for variants of F % if, given a sample in which the preselected sequence variant is present as at least F % of the sequences in the sample, the method can detect the preselected sequence at a preselected confidence of C %, S % of the time. By way of example, a method has a sensitivity of 90% for variants of 5% if, given a sample in which the preselected variant sequence is present as at least 5% of the sequences in the sample, the method can detect the preselected sequence at a preselected confidence of 99%, 9 out of 10 times (F=5%; C=99%; S=90%). Exemplary sensitivities include at least 50, 60, 70, 80, 90, 95, 98, and 99%.

“Single base substitutions” or “SBS” are defined as a replacement of a single nucleotide base with another single nucleotide base. Exemplary possible substitutions (e.g., labels): C>A, C>G, C>T, T>A, T>C, and T>G. These SBS classes can be further expanded considering the nucleotide context, e.g., considering not only the mutated base, but also the bases immediately 5′ and 3′. In some embodiments, a point mutation profile of a patient may be determined using the conventional 96 SBS mutation type classification or matrices.

As used herein, “SNVs” or “single nucleotide variants” are general terms for germline or somatic single nucleotide changes in DNA sequence.

Specificity,” as used herein, is a measure of the ability of a method to distinguish a truly occurring preselected sequence variant from sequencing artifacts or other closely related sequences. It is the ability to avoid false positive detections. False positive detections can arise from errors introduced into the sequence of interest during sample preparation, sequencing error, or inadvertent sequencing of closely related sequences like pseudo-genes or members of a gene family. A method has a specificity of X % if, when applied to a sample set of N_{Total} sequences, in which X_{True} sequences are truly variant and X_{Not true} are not truly variant, the method selects at least X % of the not truly variant as not variant. E.g., a method has a specificity of 90% if, when applied to a sample set of 1,000 sequences, in which 500 sequences are truly variant and 500 are not truly variant, the method selects 90% of the 500 not truly variant sequences as not variant. Exemplary specificities include at least 50, 60, 70, 80, 90, 95, 98, and 99%.

### Biological Sample Collection and Preparation

The methods of the present technology are useful in detecting hypermutator Pseudomonas species by detecting a mutational signature (e.g., DNA mismatch repair-deficit (dMMR) mutational signature) of the Pseudomonas species in a biological sample obtained from a subject. Samples for pathogenic Pseudomonas species detection may also comprise cultures of bacterial isolates grown on appropriate media to form colonies, wherein the cultures were prepared from a biological sample obtained from a subject.

The methods disclosed herein are useful in detecting hypermutator Pseudomonas species in biological samples derived from sterile and/or non-sterile sites. “Sterile sites” include body fluids such as whole blood, plasma, cell free plasma, urine, cerebrospinal fluid, synovial fluid, pleural fluid, pericardial fluid, intraocular fluid, tissue biopsies or endrotracheal aspirates. As used herein, “cell-free plasma” refers to plasma containing less than 1% cells by volume. “Non-sterile sites” include sputum, stool, skin swabs, inguinal swabs, nasal swabs and throat swabs. In some embodiments, the biological samples comprise skin tissue, throat swabs, stool, urine, blood, lung tissue, stomach tissue, or urinary tract tissue.

A biological sample may be suspected of containing hypermutator Pseudomonas species and/or nucleic acids of one or more hypermutator Pseudomonas species. In addition, a biological sample may be obtained from a subject suspected of being infected with one or more hypermutator Pseudomonas species. In some embodiments, the detection methods disclosed herein will be effective if used on isolated nucleic acids purified from a biological sample according to any methods well known to those of skill in the art. If desired, the sample may be collected or concentrated by centrifugation and the like. The cells of the sample may be subjected to lysis, such as by treatments with enzymes, heat surfactants, ultrasonication or a combination thereof. Alternatively, a biological sample may be processed using a commercially available nucleic acid extraction kit.

### NGS Platforms

The diagnostic methods of the present technology involve sequencing Pseudomonas nucleic acids (e.g., DNA) isolated from a biological sample obtained from the subject. In some embodiments, high throughput, massively parallel sequencing employs sequencing-by-synthesis with reversible dye terminators. In other embodiments, sequencing is performed via sequencing-by-ligation. In yet other embodiments, sequencing is single molecule sequencing. Examples of Next Generation Sequencing techniques include, but are not limited to pyrosequencing, Reversible dye-terminator sequencing, SOLiD sequencing, Ion semiconductor sequencing, Helioscope single molecule sequencing etc.

The Ion Torrent™ (Life Technologies, Carlsbad, CA) amplicon sequencing system employs a flow-based approach that detects pH changes caused by the release of hydrogen ions during incorporation of unmodified nucleotides in DNA replication. For use with this system, a sequencing library is initially produced by generating DNA fragments flanked by sequencing adapters. In some embodiments, these fragments can be clonally amplified on particles by emulsion PCR. The particles with the amplified template are then placed in a silicon semiconductor sequencing chip. During replication, the chip is flooded with one nucleotide after another, and if a nucleotide complements the DNA molecule in a particular microwell of the chip, then it will be incorporated. A proton is naturally released when a nucleotide is incorporated by the polymerase in the DNA molecule, resulting in a detectable local change of pH. The pH of the solution then changes in that well and is detected by the ion sensor. If homopolymer repeats are present in the template sequence, multiple nucleotides will be incorporated in a single cycle. This leads to a corresponding number of released hydrogens and a proportionally higher electronic signal.

The 454™ GS FLX™ sequencing system (Roche, Germany), employs a light-based detection methodology in a large-scale parallel pyrosequencing system. Pyrosequencing uses DNA polymerization, adding one nucleotide species at a time and detecting and quantifying the number of nucleotides added to a given location through the light emitted by the release of attached pyrophosphates. For use with the 454™ system, adapter-ligated DNA fragments are fixed to small DNA-capture beads in a water-in-oil emulsion and amplified by PCR (emulsion PCR). Each DNA-bound bead is placed into a well on a picotiter plate and sequencing reagents are delivered across the wells of the plate. The four DNA nucleotides are added sequentially in a fixed order across the picotiter plate device during a sequencing run. During the nucleotide flow, millions of copies of DNA bound to each of the beads are sequenced in parallel. When a nucleotide complementary to the template strand is added to a well, the nucleotide is incorporated onto the existing DNA strand, generating a light signal that is recorded by a CCD camera in the instrument.

Sequencing technology based on reversible dye-terminators: DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

Helicos's single-molecule sequencing uses DNA fragments with added polyA tail adapters, which are attached to the flow cell surface. At each cycle, DNA polymerase and a single species of fluorescently labeled nucleotide are added, resulting in template-dependent extension of the surface-immobilized primer-template duplexes. The reads are performed by the Helioscope sequencer. After acquisition of images tiling the full array, chemical cleavage and release of the fluorescent label permits the subsequent cycle of extension and imaging.

Sequencing by synthesis, like the “old style” dye-termination electrophoretic sequencing, relies on incorporation of nucleotides by a DNA polymerase to determine the base sequence. A DNA library with affixed adapters is denatured into single strands and grafted to a flow cell, followed by bridge amplification to form a high-density array of spots onto a glass chip. Reversible terminator methods use reversible versions of dye-terminators, adding one nucleotide at a time, detecting fluorescence at each position by repeated removal of the blocking group to allow polymerization of another nucleotide. The signal of nucleotide incorporation can vary with fluorescently labeled nucleotides, phosphate-driven light reactions and hydrogen ion sensing having all been used. Examples of sequencing by synthesis platforms include Illumina GA and HiSeq 2000. The MiSeq® personal sequencing system (Illumina, Inc.) also employs sequencing by synthesis with reversible terminator chemistry.

In contrast to the sequencing by synthesis method, the sequencing by ligation method uses a DNA ligase to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method is primarily used by Life Technologies' SOLiD™ sequencers. Before sequencing, the DNA is amplified by emulsion PCR. The resulting beads, each containing only copies of the same DNA molecule, are deposited on a solid planar substrate.

SMRT™ sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring at the bottom of the well is detected. The fluorescent label is detached from the nucleotide at its incorporation into the DNA strand, leaving an unmodified DNA strand.

### Diagnostic and Therapeutic Methods of the Present Technology

In one aspect, the present disclosure provides a method for treating a hypermutator Pseudomonas infection in a subject in need thereof comprising administering to the subject an effective amount of an antipseudomonal small molecule inhibitor and an effective amount of an antipseudomonal cationic peptide. In another aspect, the present disclosure provides a method for preventing hypermutator Pseudomonas-induced antibiotic resistance in a subject having a hypermutator Pseudomonas infection comprising administering to the subject an effective amount of an antipseudomonal small molecule inhibitor and an effective amount of an antipseudomonal cationic peptide. In some embodiments, the antipseudomonal cationic peptide comprises one or more of polymyxin B, D-CONGA, D-CONGA-Q7 or colistin. Additionally or alternatively, in certain embodiments, the antipseudomonal small molecule inhibitor comprises one or more of monobactams, fluoroquinolines, cephalosporins or carbapenems. Examples of monobactams include, but are not limited to, aztreonam, azactam, tigemonam, nocardicin A, tabtoxin, and cayston. Examples of fluoroquinolines include, but are not limited to, ciprofloxacin, gemifloxacin, levofloxacin, delafloxacin, gemifloxacin, moxifloxacin, norfloxacin, and ofloxacin. Examples of cephalosporins include, but are not limited to, cephalexin, cefadroxil, cephradine, cephalexin, cefazolin, cefuroxime, cefprozil, loracarbef, cefuroxime, cefoxitin, cefotetan, ceftriaxone, cefdinir, cefixime, cefpodoxime, cefditoren, ceftibuten, cefdinir, ceftazidime, cefotaxime, cefoperazone, ceftizoxime, cefepime, ceftaroline, ceftolozane, or cefiderocol. Examples of carbapenems include, but are not limited to, doribax, doripenem, ertapenem, imipenem, cilastatin, relebactam, invanz, meropenem, vaborbactam, merrem IV, primaxin, recarbrio, sulopenem, sulopenem etzadroxil/probenecid, or vabomere.

In any of the preceding embodiments of the methods disclosed herein, the Pseudomonas infection is caused by a Pseudomonas species selected from among P. aeruginosa, P. fluorescens, P. putida, P. cepacia, P. stutzeri, P. maltophilia, and P. putrefaciens. In certain embodiments, the Pseudomonas infection is caused by P. aeruginosa. Additionally or alternatively, in some embodiments, the subject is diagnosed with or is at risk for cystic fibrosis or an acute infection. The acute infection may comprise a pressure sore infection, a burn infection, a wound infection or bloodstream infection.

In any and all embodiments of the methods disclosed herein, the antipseudomonal small molecule inhibitor and the antipseudomonal cationic peptide is administered separately, sequentially, or simultaneously. Additionally or alternatively, in some embodiments, the antipseudomonal small molecule inhibitor or the antipseudomonal cationic peptide is administered orally, intravenously, intramuscularly, intraperitoneally, or subcutaneously.

In one aspect, the present disclosure provides a method for detecting a hypermutator Pseudomonas infection in a subject comprising (a) sequencing Pseudomonas nucleic acids isolated from a biological sample obtained from the subject; (b) generating a mutation spectrum of the Pseudomonas nucleic acid sequences; and (c) detecting the presence of a hypermutator Pseudomonas infection when a Pseudomonas DNA mismatch repair-deficit (dMMR) mutational signature is detected in the mutation spectrum. In another aspect, the present disclosure provides a method for predicting the risk of antibiotic resistance in a subject having a Pseudomonas infection comprising (a) sequencing Pseudomonas nucleic acids isolated from a biological sample obtained from the subject; (b) generating a mutation spectrum of the Pseudomonas nucleic acid sequences; and (c) determining that the subject is at risk for antibiotic resistance when a Pseudomonas DNA mismatch repair-deficit (dMMR) mutational signature is detected in the mutation spectrum. The Pseudomonas nucleic acid sequences may comprise genomic DNA or cell-free DNA. Additionally or alternatively, in certain embodiments, the dMMR mutational signature comprises an increase in C>T transitions in NCC and NCG contexts and/or an increase in T>C transitions in CTN and GTN contexts. In some embodiments, the dMMR mutational signature comprises an increase in C>T transitions in ACC, GCC, TCC, CCC, ACG, GCG, TCG, and/or CCG contexts. Additionally or alternatively, in certain embodiments, the dMMR mutational signature comprises an increase in T>C transitions in CTA, CTG, CTC, CTT, GTA, GTC, GTG, and/or GTT. In some embodiments, the Pseudomonas DNA mismatch repair-deficit (dMMR) mutational signature is detected by using a cosine similarity of the comparison between the mutation spectrum of Pseudomonas nucleic acid sequences obtained from the biological sample with a mutation spectrum of nucleic acid sequences obtained from a control hypermutator Pseudomonas reference strain. The biological sample may comprise skin tissue, throat swabs, stool, urine, blood, lung tissue, stomach tissue, or urinary tract tissue. In certain embodiments, the methods of the present technology further comprise administering to the subject an effective amount of an antipseudomonal small molecule inhibitor and an effective amount of an antipseudomonal cationic peptide.

In one aspect, the present disclosure provides a method for preventing multi-drug resistance in a subject infected with Pseudomonas comprising (a) sequencing Pseudomonas nucleic acids isolated from a biological sample obtained from the subject; (b) generating a mutation spectrum of the Pseudomonas nucleic acid sequences; (c) detecting a Pseudomonas DNA mismatch repair-deficit (dMMR) mutational signature in the mutation spectrum; and (d) administering to the subject an effective amount of an antipseudomonal small molecule inhibitor and an effective amount of an antipseudomonal cationic peptide. In another aspect, the present disclosure provides a method for selecting an antipseudomonal therapy fora subject having a Pseudomonas infection comprising (a) sequencing Pseudomonas nucleic acids isolated from a biological sample obtained from the subject; (b) generating a mutation spectrum of the Pseudomonas nucleic acid sequences; (c) detecting a Pseudomonas DNA mismatch repair-deficit (dMMR) mutational signature in the mutation spectrum; and (d) administering to the subject an effective amount of an antipseudomonal small molecule inhibitor and an effective amount of an antipseudomonal cationic peptide. The Pseudomonas nucleic acid sequences may comprise genomic DNA or cell-free DNA.

Additionally or alternatively, in certain embodiments, the dMMR mutational signature comprises an increase in C>T transitions in NCC and NCG contexts and/or an increase in T>C transitions in CTN and GTN contexts. In some embodiments, the dMMR mutational signature comprises an increase in C>T transitions in ACC, GCC, TCC, CCC, ACG, GCG, TCG, and/or CCG contexts. Additionally or alternatively, in certain embodiments, the dMMR mutational signature comprises an increase in T>C transitions in CTA, CTG, CTC, CTT, GTA, GTC, GTG, and/or GTT. In some embodiments, the Pseudomonas DNA mismatch repair-deficit (dMMR) mutational signature is detected by using a cosine similarity of the comparison between the mutation spectrum of Pseudomonas nucleic acid sequences obtained from the biological sample with a mutation spectrum of nucleic acid sequences obtained from a control hypermutator Pseudomonas reference strain.

In any of the preceding embodiments of the methods disclosed herein, Pseudomonas nucleic acids are sequenced via whole genome sequencing (WGS).

In any of the preceding embodiments of the methods disclosed herein, the Pseudomonas infection is caused by a Pseudomonas species selected from among P. aeruginosa, P. fluorescens, P. putida, P. cepacia, P. stutzeri, P. maltophilia, and P. putrefaciens. In some embodiments, the antipseudomonal cationic peptide comprises one or more of polymyxin B, D-CONGA, D-CONGA-Q7 or colistin. Additionally or alternatively, in certain embodiments, the antipseudomonal small molecule inhibitor comprises one or more of monobactams, fluoroquinolines, cephalosporins, or carbapenems. Examples of monobactams include, but are not limited to, aztreonam, azactam, tigemonam, nocardicin A, tabtoxin, and cayston. Examples of fluoroquinolines include, but are not limited to, ciprofloxacin, gemifloxacin, levofloxacin, delafloxacin, gemifloxacin, moxifloxacin, norfloxacin, and ofloxacin. Examples of cephalosporins include, but are not limited to, cephalexin, cefadroxil, cephradine, cephalexin, cefazolin, cefuroxime, cefprozil, loracarbef, cefuroxime, cefoxitin, cefotetan, ceftriaxone, cefdinir, cefixime, cefpodoxime, cefditoren, ceftibuten, cefdinir, ceftazidime, cefotaxime, cefoperazone, ceftizoxime, cefepime, ceftaroline, ceftolozane, or cefiderocol. Examples of carbapenems include, but are not limited to, doribax, doripenem, ertapenem, imipenem, cilastatin, relebactam, invanz, meropenem, vaborbactam, merrem IV, primaxin, recarbrio, sulopenem, sulopenem etzadroxil/probenecid, or vabomere.

In any and all embodiments of the methods disclosed herein, the mutation spectrum comprises a trinucleotide mutation spectrum of SNVs present in the Pseudomonas nucleic acid sequences.

### EXAMPLES

The present technology is further illustrated by the following Examples, which should not be construed as limiting in any way.

### Example 1: Materials and Methods

### Bacterial Strains and Growth Conditions

Wild type, parent strain MPAO1 and transposon mutants MPAO1-ΔmutS (strain PW7149) and MPAO1-ΔmutL (strain PW5709), were purchased from the Pseudomonas aeruginosa two allele transposon library of Colin Manoil, PhD at the University of Washington (funded by grant no. NIH P30 DK089507) (51). P. aeruginosa strains were initially streaked on Pseudomonas Isolation Agar (PIA) (purchased from BD Difco). All strains were cultured in Luria Bertani (LB) broth (Miller) (purchased from VWR Life Sciences) at 37° C. at 220 rpm for 18 hours to make glycerol frozen stocks stored at −80° C. prior to experimentation.

### Confirmation of Insertional Transposon Knockout Mutants

Insertional transposon mutants were confirmed via polymerase chain reaction (PCR) with gene-flanking and transposon-annealing primer sets (Table 1). Primer sets were first optimized on MPAO1. PCR products were visualized on 1% agarose gels via electrophoresis and mutant expected sizes were confirmed (expected sizes in Table 1).

TABLE 1
Sequences of PCR primers flanking gene insertion site
and annealing to insertional transposon used to confirm
insertional transposon mutagenesis in MPAO1-ΔmutS and
MPAO1-ΔmutL, along with expected size for each product.
“Gene” refers to FWD and REV primers flanking the
target gene for insertional transposition, whereas
“transposon” refers to a primer annealing to the
inserted transposon sequence (direction of transposon)
and a primer flanking gene insertion site to confirm
insertion. All sequences are written 5′→3′.

| | | | | KO- | WT |
|---|---|---|---|---|---|
| | | FWD primer | REV primer | size | size |
| MPAO1- | Gene | CTGGATATCAACC | GATTCAGGTCGAG | No | 876 |
| ΔmutS | | TCAGCGG | GTTCAGC | band | bp |
| (PW7149) | | (SEQ ID NO: 1) | (SEQ ID NO: 2) | | |
| — | Transposon | GGGTAACGCCAGG | CTGGATATCAACCT | 554 | No |
| | | GTTTTCC | CAGCGG | bp | band |
| | | (SEQ ID NO: 3) | (SEQ ID NO: 4) | | |
| MPAO1- | Gene | GTCGCGGATATCG | AAGGGCATCTACA | No | 813 |
| ΔmutL | | ATCAGG | TTCTCGC | band | bp |
| (PW5709) | | (SEQ ID NO: 5) | (SEQ ID NO: 6) | | |
| — | Transposon | GGGTAACGCCAGG | AAGGGCATCTACA | 267 | No |
| | | GTTTTCC | TTCTCGC | bp | band |
| | | (SEQ ID NO: 7) | (SEQ ID NO: 8) | | |

### Rifampicin Reversion Mutant Frequency Assays

Bacteria were streaked from glycerol stocks on LB agar, and independent triplicate colonies were inoculated into 10 mL Mueller-Hinton (MH) broth (purchased from BD BBL) and incubated for 18 hours overnight. 1 mL of each culture was pelleted at 4,000×g for 10 minutes, and the pellet was washed with sterile 1× phosphate buffered saline (PBS) 3 times. The pellet was resuspended in 1 mL 1×PBS and serially diluted 10-fold in 1×PBS. Dilutions were spotted (10 μL) and spread (100 μL) on MH agar and MH agar containing 100 μg/mL rifampicin and incubated at 37° C. for 24 hours. Mutant frequency was determined by mutants (colonies grown on MH+rifampicin)/viable cells (colonies grown on MH) (13).

### Antimicrobial Peptide Preparation

D-CONGA and D-CONGA-Q7 were synthesized using Fmoc solid-phase chemistry and purified to >95% via high performance liquid chromatography by Bio-synthesis Inc, with identity confirmed via MALDI mass spectrometry. Solutions were prepared by dissolving desired mass into 0.025% (v/v) acetic acid in water, and peptide concentration was determined by absorbance at 280 nm.

### Antimicrobial Susceptibility Testing

MICs were determined via microbroth dilution assay in 96-well culture plates with 2-fold serial dilutions of tested antibiotic in Mueller-Hinton (cation-adjusted) broth, inoculated with 50 μL of 2.75×105 CFU/mL of each bacterial strain and incubated at 37° C., 200 rpm for 24 hours. MIC experiments were performed using biological triplicates.

### In Vitro Adaptive Evolution

Serial passaging of bacterial strains was performed in 96-well culture plates with 2-fold serial dilutions of antibiotic in MH broth, inoculated with 50 μL 2.75×105 CFU/mL of each strain and incubated at 37° C., 200 rpm for 24 hours. Next, bacteria taken from the well with the highest concentration of antibiotic that still exhibited growth (compared to negative control with no inoculum) was diluted 1:100 into 5 mL of fresh LB containing no antibiotics and grown overnight. The bacteria were then serially passaged in the same antibiotic and MIC determined for all antibiotics (aztreonam, ciprofloxacin, D-CONGA, D-CONGA-Q7, polymyxin B). See Antimicrobial susceptibility testing section. The bacteria were serially passaged a total of 10 times.

### DNA Purification

Aliquoted cultures of evolved clones from each lineage were stored at −80° C. after time of emergence of resistance and experimental endpoint. Frozen aliquoted cultures were thawed and pelleted (500 μL) at 4,000×g for 10 minutes, and the supernatant was discarded. Genomic DNA was isolated using a Qiagen DNeasy Blood and Tissue Kit per manufacturer's instructions. DNA quality and concentration was determined via absorbance at 260 nm, 280 nm, and 230 nm.

### Whole Genome Sequencing and Variant Calling

Genomic DNA preps from desired resistant and control lineages were whole genome sequenced using Illumina short-read sequencing (NextSeq 2000). Sample libraries were prepared using Illumina DNA Prep Kit and IDT 10 bp UDI indices. Demultiplexing, quality control (only reads with Q>30 kept), and adapter trimming were performed using Illumina bcl-convert (v3.9.3). Each sample produced a minimum of 400 Mbp high quality reads (2×151 bp), with an average depth of coverage of 150× of the ~6.3 Mbp genome.

Paired end reads were aligned to the PAO1 reference genome (NCBI accession #NC_002516.2) and variants were called to reference using breseq (v0.36.1) (52). All variants called were at 100% frequency unless otherwise specified. Variants that were common to the parental strain prior to adaptive evolution and that were common among all lineages with the same parental independent colony (i.e. common among all colony 1 lineages, so assumed to be in colony 1 prior to evolution) were excluded, leaving only candidate de novo mutations. Common de novo mutations among independent lineages under same treatment were identified as candidate resistance-conferring mutations.

### Mutational Signature Analysis

Variant call format (VCF) files containing de novo mutations from each sample were loaded into and parsed in R using the tidyverse suite (53). For each mutation, the reference base was retrieved from the PAO1 reference genome sequence along with flanking reference bases on both the 3′ and 5′ ends to produce the trinucleotide mutation context. Where necessary, trinucleotide contexts were converted to their reverse complement to reflect one of the canonical 6 types of bases changes (C>{A,G,T} or T>{A,C,G}). Individual mutation spectra were plotted in their 96-trinucleotide context. Due to few total detected de novo mutations, all mutations from MPAO1-ΔmutS samples were also compiled into one signature. Established human (hg38) MMR-deficiency-associated mutational signatures were retrieved from the Catalogue of Somatic Mutations in Cancer (COSMIC; SBS6/15/21/26/44) (54). Human MMR signatures were also additively combined into a compiled signature. Cosine similarities between the mutation distributions of relevant samples and the COSMIC MMR-associated signatures were calculated using via the R package MutationalPatterns (55).

### Lipid A Extraction and MALDI-MS Analysis

1 μL of sample pellet was scraped and plated directly to a steel re-usable MALDI plate. 1 μL of 70% citric acid extraction buffer was spotted on top of the plated bacteria. The steel MALDI plate was added to chamber with water on bottom and placed in 110 degree Celsius oven for 30 minutes. After, the plate was rinsed with water and air dried. 1 μL of Norharmane matrix was spotted on each sample. Samples were analyzed in negative ion mode on a Bruker Microflex in negative ion mode. Data were processed with flexAnalysis software.

Sputum Collection and Processing from People with Cystic Fibrosis (pwCF)

Subjects with CF and known history of P. aeruginosa respiratory colonization were recruited from Tulane University Medical Center (Tulane IRB 2019-1840). After obtaining informed consent, spontaneous sputum samples were collected in sterile screw-cap cups for processing. Samples were processed within 24 hours of collection, kept at 4° C. overnight if needed or processed directly from room temperature if within 4 hours of collection.

Sputum samples were processed with equal volume 6.5 mM DTT, vortexed for 45 seconds and rotated for 30 minutes. Processed sputum was streaked onto PIA for P. aeruginosa isolation. Colonies with distinct morphotypes were collected as different strains from each patient sample and pure cultured on PIA. Human lung tissues were obtained from 4 patients undergoing pulmonary resections. Normal tissue was obtained from outside the tumor margin, as determined by intraoperative pathology examination. The human lung tissues were dissociated using a similar protocol as for the mice. Briefly, the lung tissue was minced into small cubic pieces and washed several times by pre-cooled PBS. Excessive PBS was drained and tissues were further minced and dissociated in 8-10 volumes of digestion buffer for 1-1.5 hours. Dissociated tissues were processed as described above and cell pellets were resuspended in 2% HI-FBS S-MEM for antibody staining.

### Statistical Analyses

All resistance acquisition data was analyzed in units of fold-change to normalize for differences in starting MIC across strains. Fold-change in MIC data for all strains and treatment groups was natural log-transformed and simple linear regression was performed considering each replicate fold-change value as an individual point. Slope, standard error of slope, and total number of values of each treatment group of interest were analyzed via unpaired t-test (ΔmutS vs WT) or ordinary one-way ANOVA (cross-resistance acquisition of ΔmutS+AB vs −AB vs WT), and α-values were corrected for multiple comparisons using the Bonferroni method. Differences in estimated mutation rate across treatment groups were determined via ordinary one-way ANOVA (Brown-Forsythe test). For all statistical tests, p<0.05 was considered significant. All figures were created and statistical tests were done using GraphPad Prism 9.3.1.

### Example 2: MMR-Deficiency, Characterized by a Hallmark Mutational Signature, Drives Drug Resistance

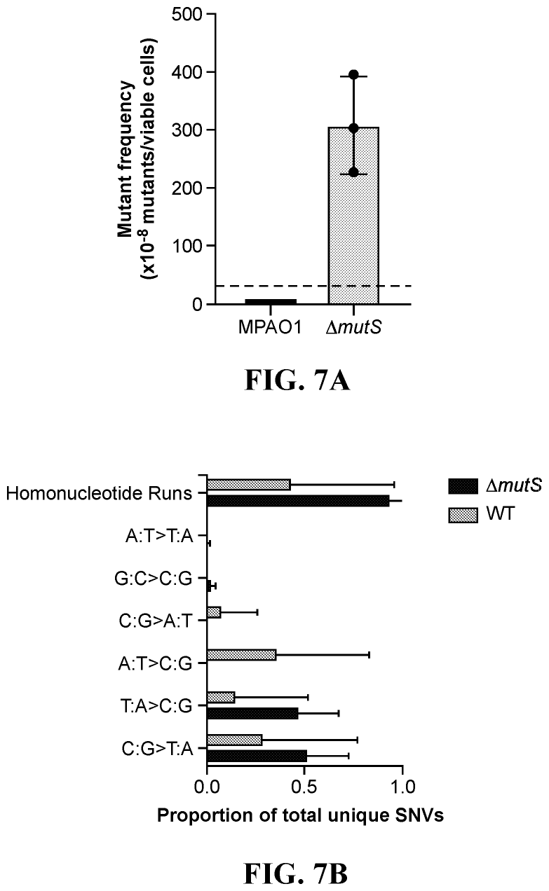

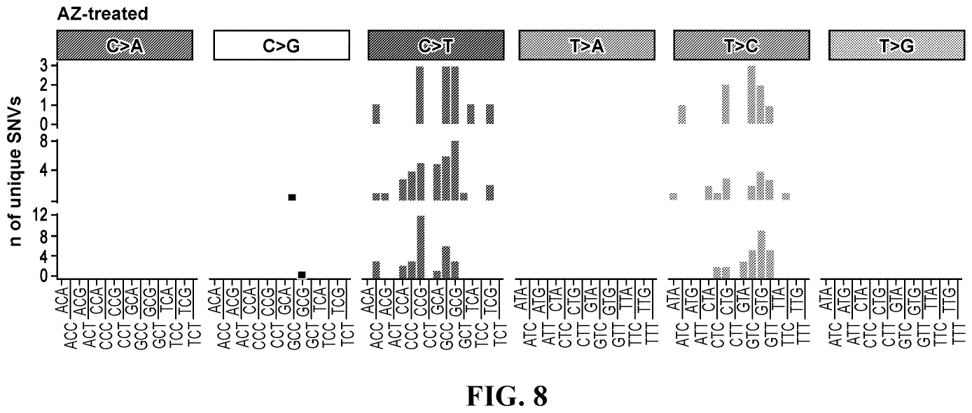

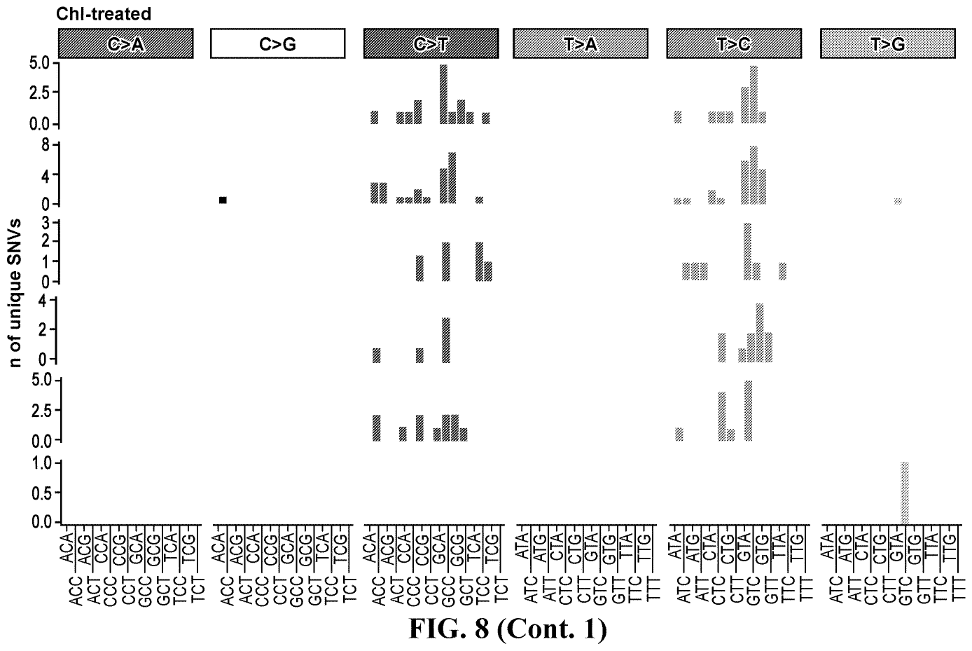

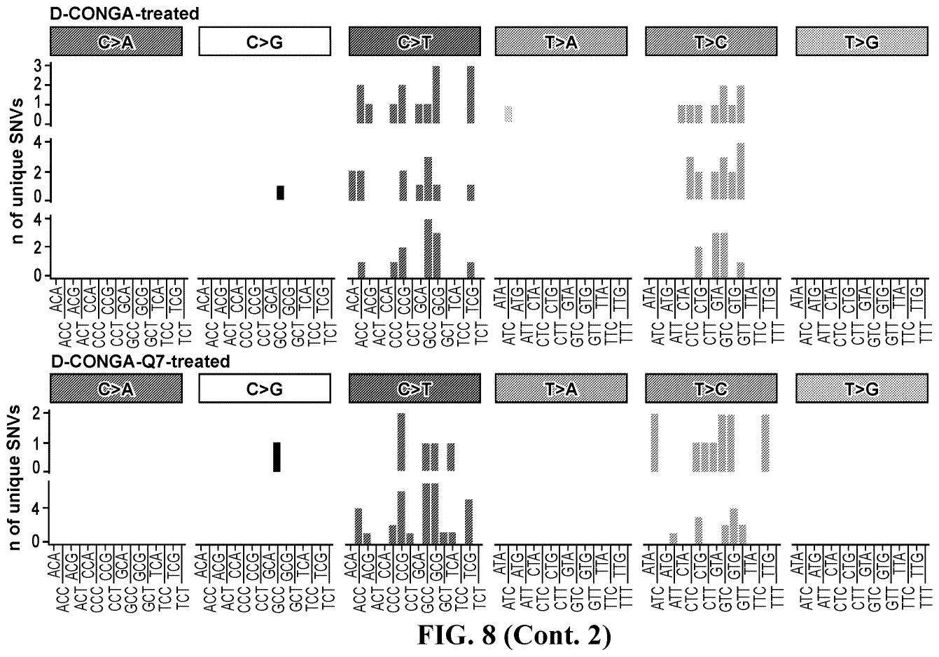

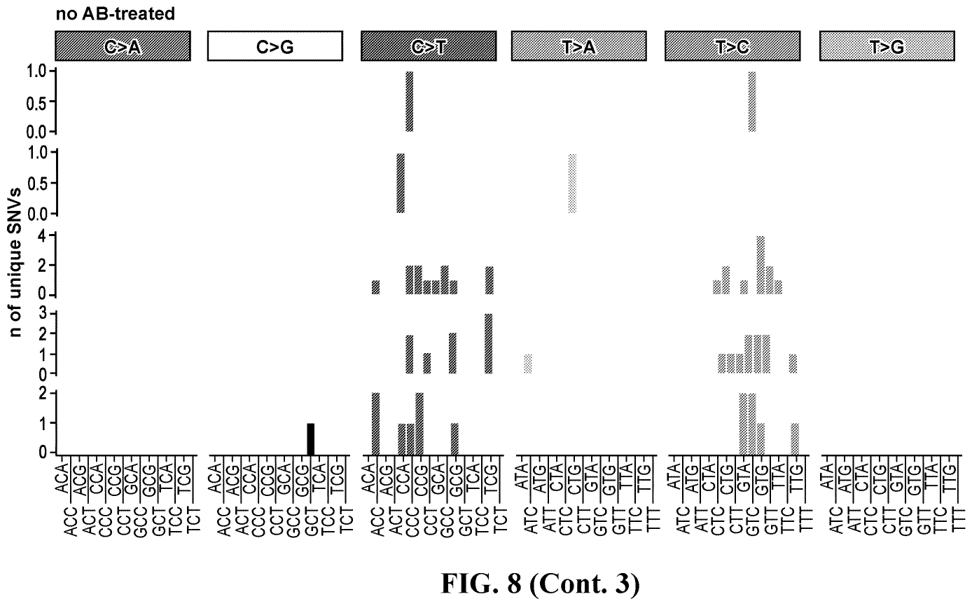

Previous work has shown MMR-deficiency enhances antibiotic resistance acquisition in P. aeruginosa in vitro and is characterized by enriched transition and indel mutations in the genome (6, 16, 17). Therefore, characterization of the full trinucleotide mutational signature in MMR-deficient P. aeruginosa laboratory strains following antibiotic resistance acquisition was performed. Wild type and mutS-deficient P. aeruginosa strain MPAO1 underwent in vitro adaptive evolution by repeated exposure to the antipseudomonal drugs commonly used to treat pwCF aztreonam and colistin (polymyxin E), along with chloramphenicol and investigational antimicrobial peptides D-CONGA and D-CONGA-Q7 (Table 2) (18-22). D-CONGA and D-CONGA-Q7 are cationic antimicrobial peptides developed via synthetic molecular evolution and are efficacious in vitro and in vivo against drug-resistant clinical isolates of Staphylococcus aureus, Klebsiella pneumoniae, Acinetobacter baumannii, and P. aeruginosa, including P. aeruginosa CF isolates (23). mutS-deficient MPAO1 was validated as a hypermutator using rifampicin reversion frequency assay prior to experimentation (FIG. 7A). Strains were susceptible to all compounds, except for chloramphenicol due to anticipated intrinsic resistance (Table 3).

TABLE 2
Description of all antimicrobial agents
used in experiments and discussed.

| Antimicrobial | Drug Class | Cell target | Nature | Abbrev. |
|---|---|---|---|---|
| Chlorampheni- | Chlorampheni- | 50S ribosome | Antibiotic | Chl |
| col | col | | | |
| Streptomycin | Aminoglycoside | 30S ribosome | Antibiotic | Str |
| Ciprofloxacin | Fluoroquinolone | DNA gyrase | Antibiotic | CIP |
| Aztreonam | Monobactam | Cell wall | Antibiotic | AZ |
| Tobramycin | Aminoglycoside | 30S ribosome | Antibiotic | TOB |
| Polymyxin B | Antimicrobial | Cell membrane | Peptide | PB |
| | peptide | | | |
| D-CONGA | Antimicrobial | Cell membrane | Peptide | N/A |
| | peptide | | | |
| D-CONGA-Q7 | Antimicrobial | Cell membrane | Peptide | N/A |
| | peptide | | | |
| Colistin | Antimicrobial | Cell membrane | Peptide | COL |
| | peptide | | | |

TABLE 3
MIC for indicated strain, average of triplicates (μg/mL).
Average (MICs) determined via antibiotic susceptibility
microbroth dilution assays of all antibiotics used for resistance
testing on all strains, along with susceptibility denotation
according to the CLSI. No susceptibility denotation is reported
for novel peptides D-CONGA and D-CONGA-Q7.

| Antimicrobial | PAO1 | | MPAO1 | | ΔmutS | | ΔmutL | |
|---|---|---|---|---|---|---|---|---|
| Chloramphenicol | 400 | (R) | ≥400 | (R) | ≥400 | (R) | 400 | (R) |
| Streptomycin | 21 | (R) | 8 | (I) | 32 | (R) | 43 | (R) |
| Ciprofloxacin | 0.25 | (S) | 0.5 | (S) | 1 | (I) | 0.3 | (S) |
| Aztreonam | 3 | (S) | 2 | (S) | 13 | (I) | 30 | (I) |
| Polymyxin B | 2 | (S) | 2 | (S) | 2 | (S) | 3 | (I) |
| Colistin | 2 | (S) | 2 | (S) | 2 | (S) | 2 | (S) |
| D-CONGA | 8 | μM | 8 | μM | 8 | μM | 4 | μM |
| D-CONGA-Q7 | 8 | μM | 8 | μM | 8 | μM | 8 | μM |

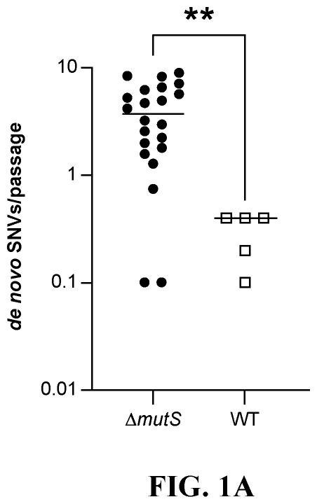

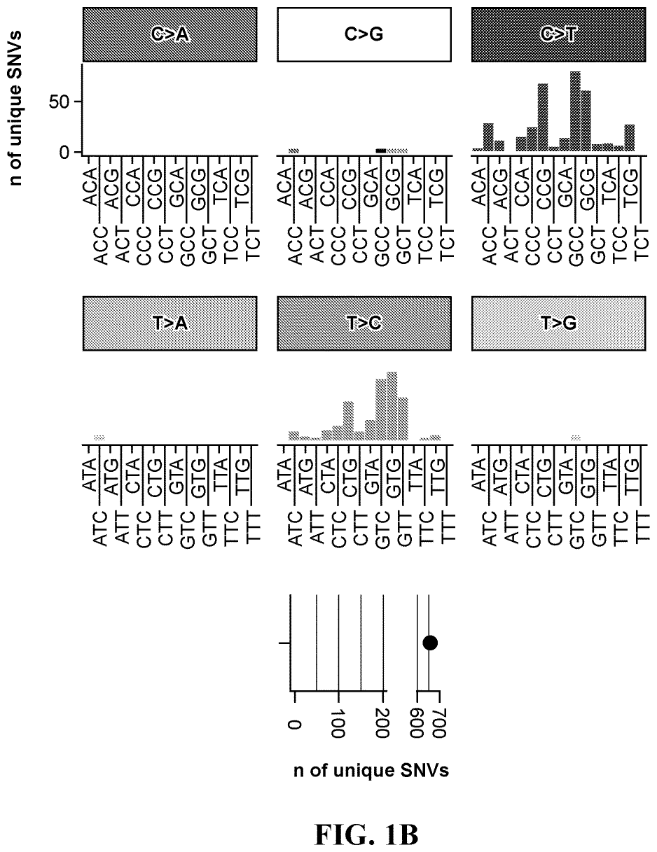

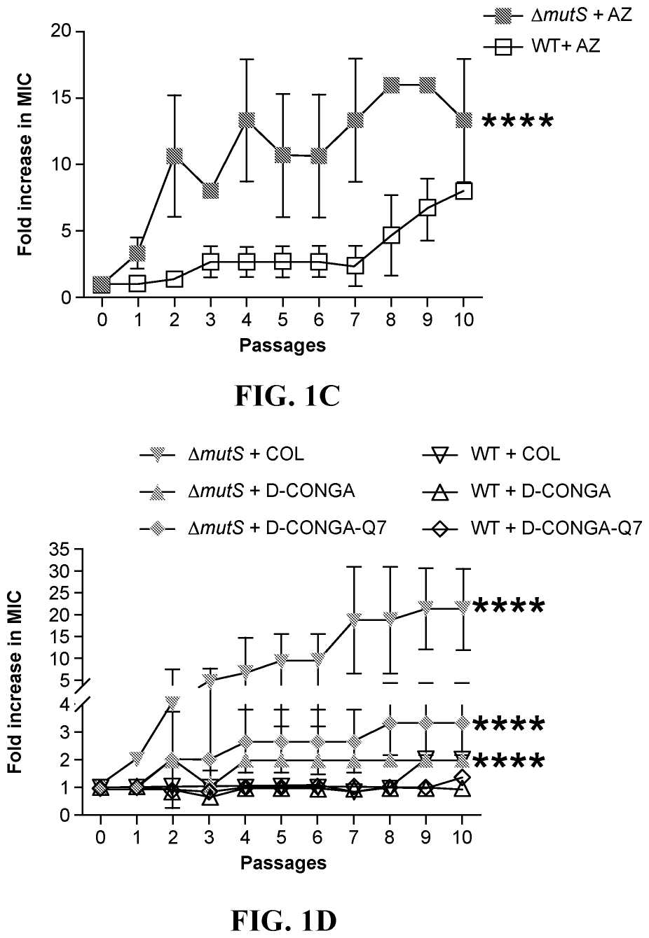

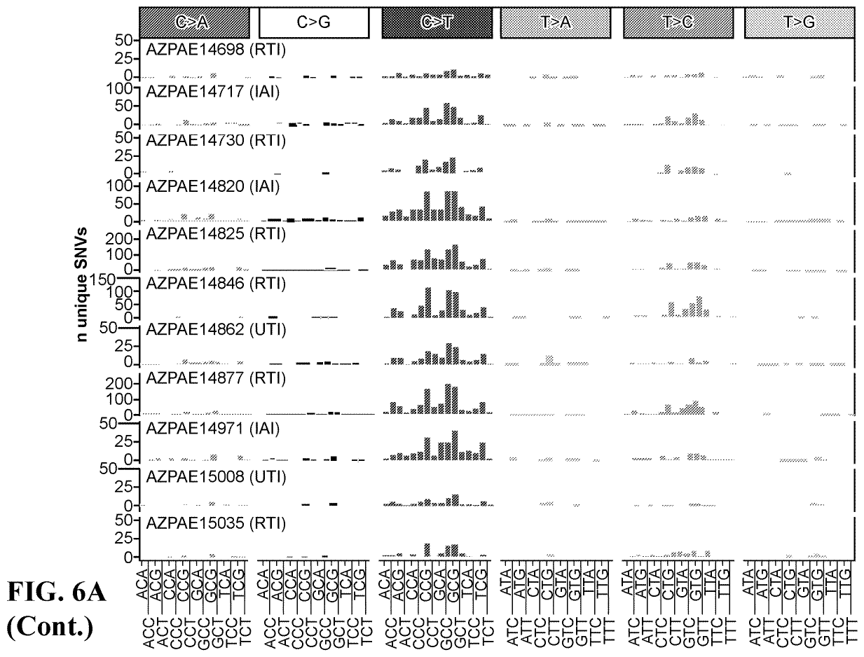

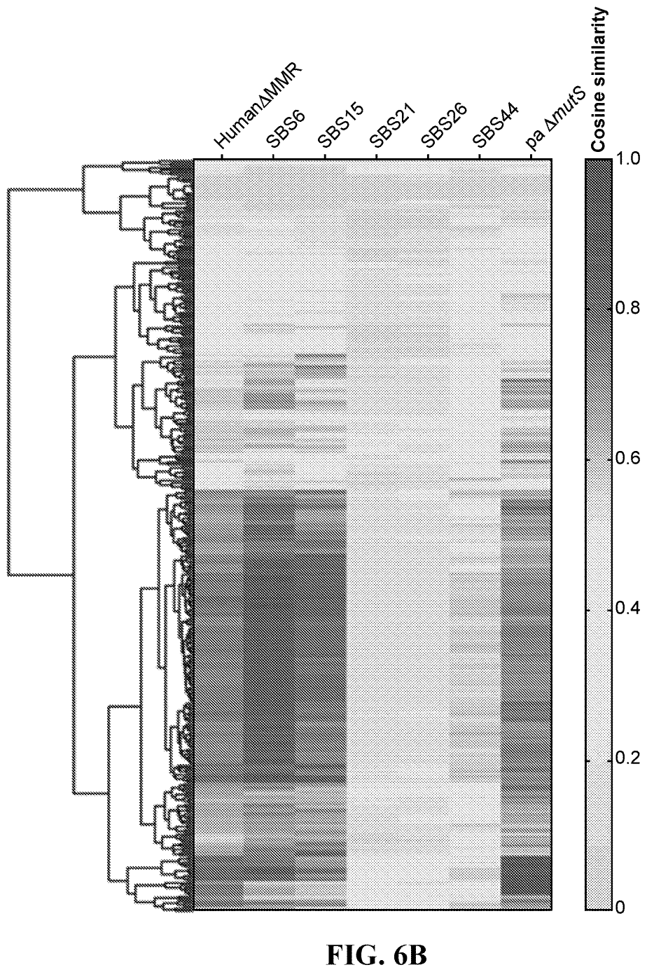

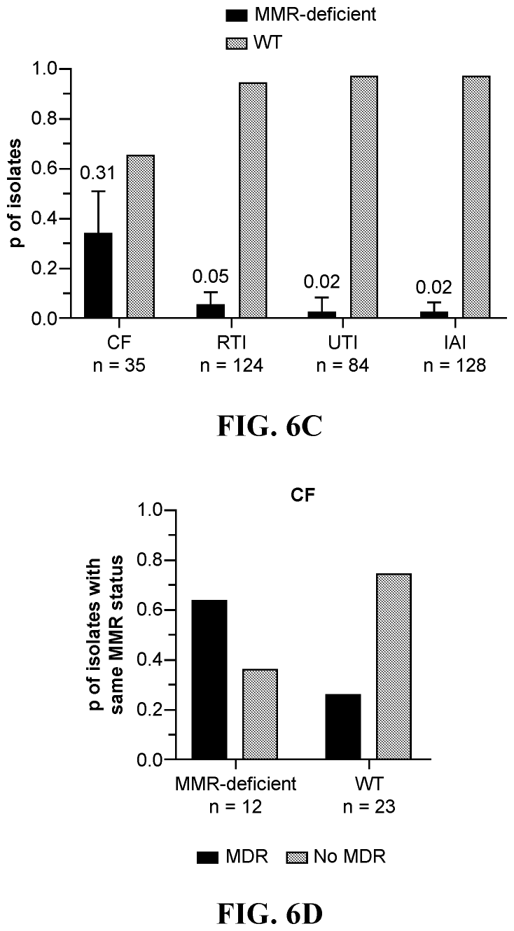

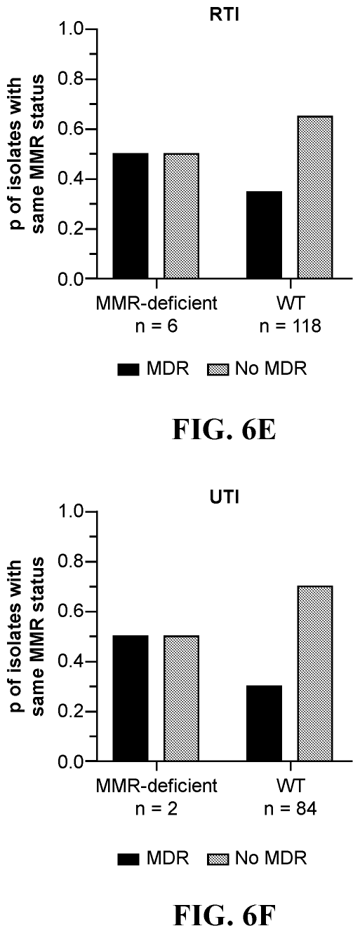

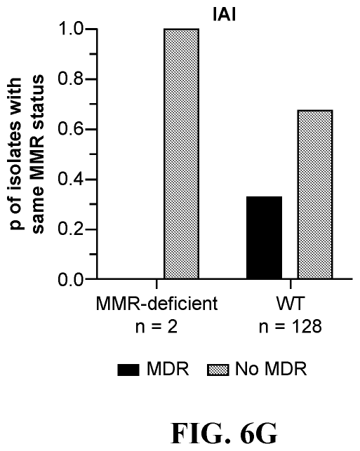

Evolved bacterial clones were subject to WGS and de novo mutation identification upon emergence of drug resistance and at the experimental endpoint. Higher mutation rates (FIG. 1A) and enriched transitions and indels in homopolymers (FIG. 7B) were observed in MPAO1-ΔmutS clones compared to MPAO1, consistent with MMR-deficiency and hypermutator phenotype. No significant shift in mutation spectra was observed due to antibiotic treatment (FIG. 6). Due to the low number of trinucleotide variants in any single clone, the observed mutations for all MPAO1-ΔmutS clones for analysis were combined (FIG. 1B). The increased C>T transitions were enriched in NCC and NCG contexts, with a 5′ preference to C and G. The increased T>C transitions were elevated in CTN and GTN contexts, particularly in GTC and GTG contexts. As these mutations are driven by MMR-deficiency, this pattern represents the de facto P. aeruginosa MMR-deficiency mutational signature.

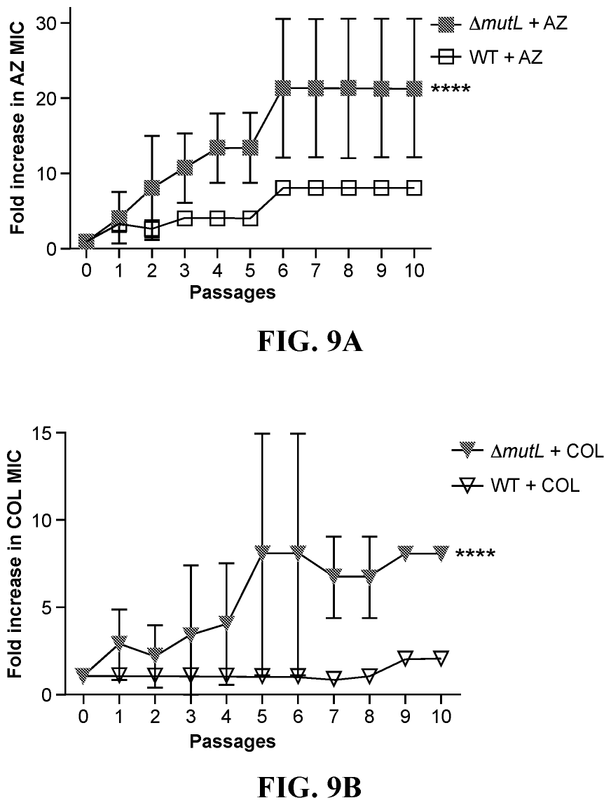

Rapid acquisition of resistance to both aztreonam (FIG. 1C) and colistin (FIG. 1D) was observed in MPAO1-ΔmutS, consistent with previous studies using hypermutator strains (16, 17). MPAO1-ΔmutL also exhibited rapid resistance acquisition, supporting MMR-deficiency as the driver (FIGS. 9A-9B). MPAO1 eventually displayed an elevated minimum inhibitory concentration (MIC) to both aztreonam and colistin, but it did so more slowly indicating that MMR-deficiency accelerated resistance acquisition. Induction of resistance to D-CONGA and D-CONGA-Q7 has not been observed for wild type P. aeruginosa (PAO1) but MPAO1-ΔmutS developed resistance to both peptides (FIG. 1D) (23). These results indicate that MMR-deficient P. aeruginosa possesses a hallmark mutational signature that is associated with more rapid emergence of drug resistance under experimental in vitro conditions.

### Example 3: Antibiotic Treatment of MMR-Deficient P. aeruginosa Induces MDR Through Shared Mechanisms of Resistance

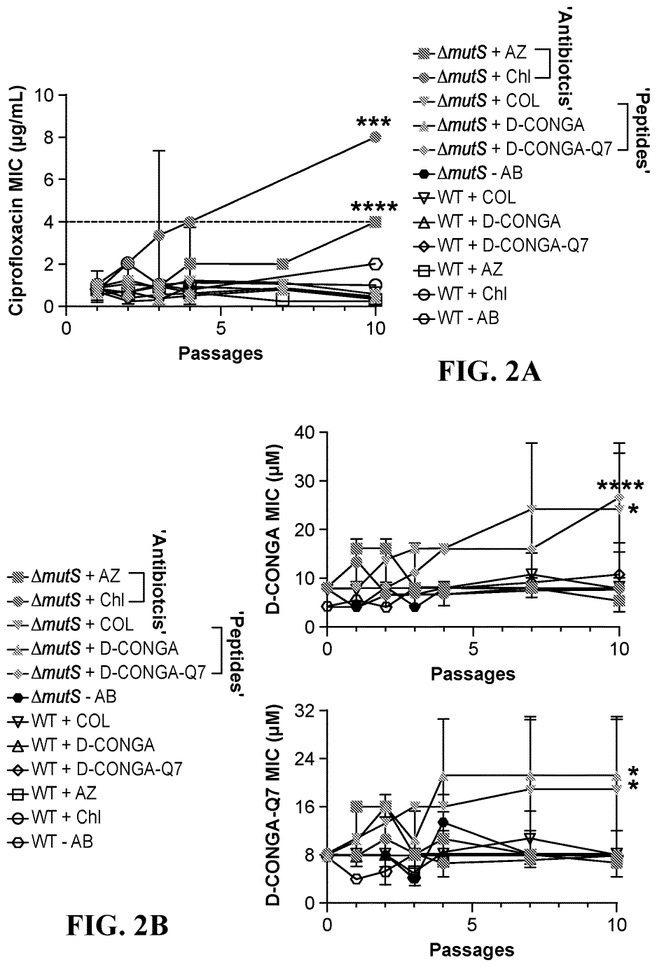

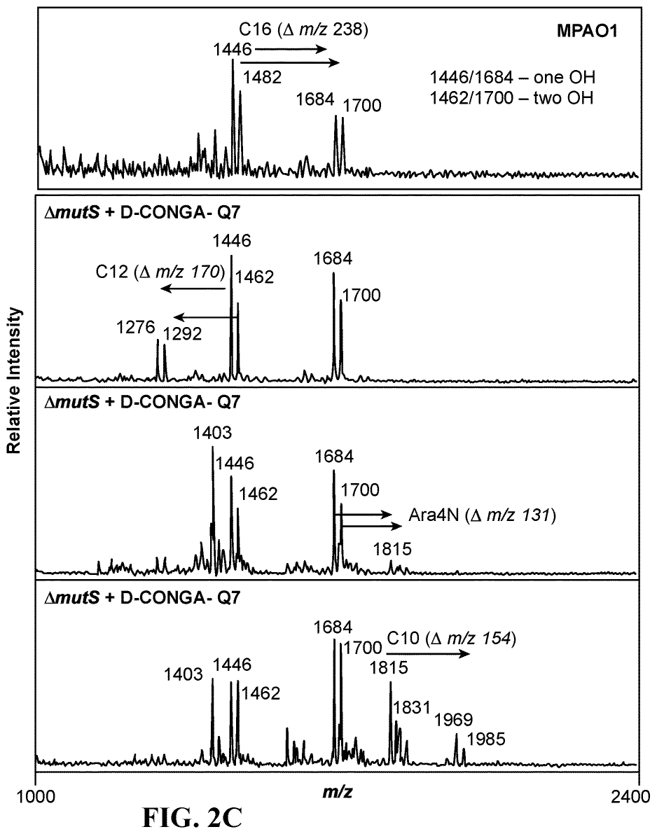

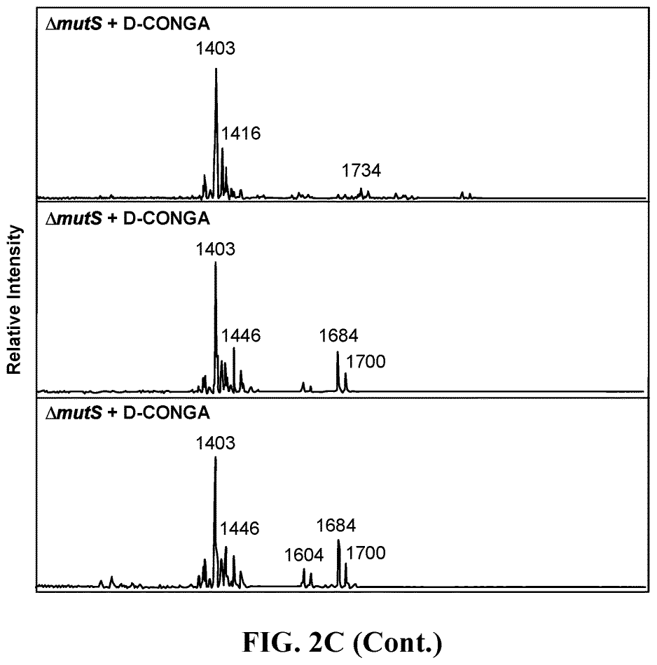

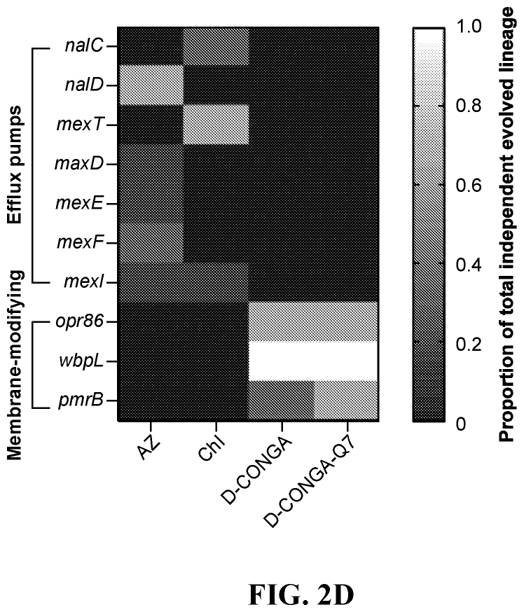

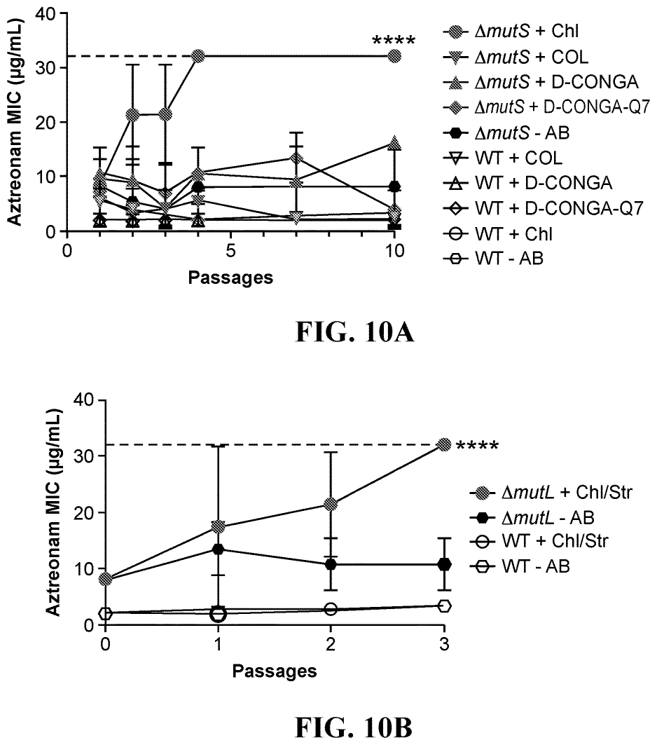

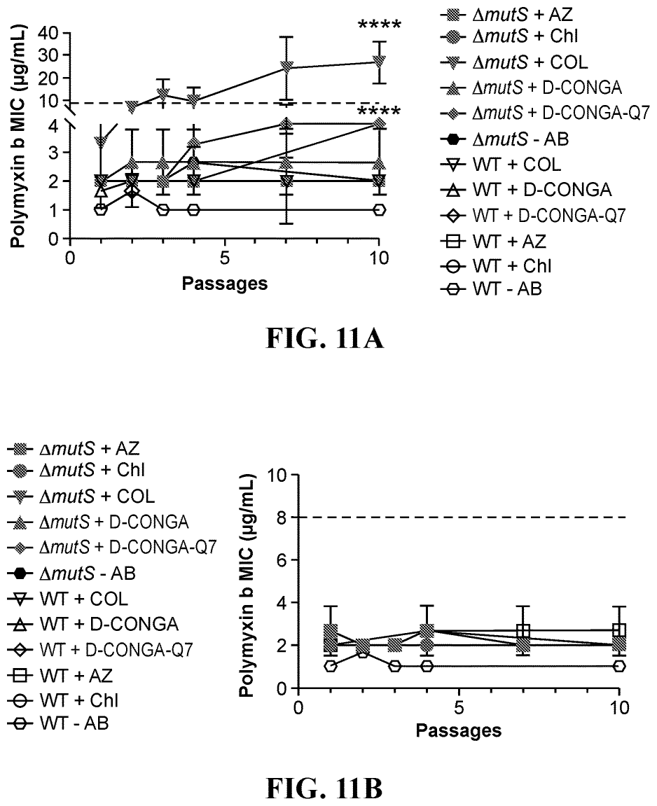

Although there is a strong association between hypermutation and MDR in P. aeruginosa clinical isolates, the mechanism of how MMR-deficiency drives MDR acquisition is not fully understood. To examine this, MICs of the evolved MPAO1-ΔmutS and MPAO1 clones (FIG. 1) were assessed against a panel of five antibiotics from three different classes (Table 2). A summary of MIC values is presented in Table 4. Treatment of MPAO1-ΔmutS with the antibiotics aztreonam or chloramphenicol induced cross-resistance to antibiotics in a different class such as ciprofloxacin (FIG. 2A and FIG. 10A), whereas no cross resistance developed in antibiotic-treated wild type MPAO1. Similarly, treatment of MPAO1-ΔmutS, but not wild type MPAO1, with colistin, D-CONGA, or D-CONGA-Q7 induced cross-resistance to other peptides (FIG. 2B and FIG. 11A). However, exposure to colistin, D-CONGA, or D-CONGA-Q7 failed to induce marked cross-resistance to antibiotics (FIG. 2A) and treatment with antibiotic compounds resulted in no change in peptide MICs (FIG. 2B and FIG. 11B). In vitro adaptive evolution of MPAO1-ΔmutL produced similar result of treatment-induced cross-resistance as MPAO1-ΔmutS, suggesting rapid MDR acquisition is characteristic of and dependent on MMR-deficiency (FIGS. 2A-2B and FIG. 10B).

TABLE 4
Summary of observed treatment-induced multidrug resistance across tested
strains. Average MICs (independent biological triplicates) of respective drug at end points
following evolution of MPAO1-ΔmutS, MPAO1-ΔmutL, Pa23, or MPAO1 in aztreonam,
chloramphenicol, colistin, D-CONGA, D-CONGA-Q7, or no antibiotic treatment. Values are
expressed as absolute MIC (fold-change relative to starting MIC before treatment). MICs of
aztreonam, ciprofloxacin, colistin, and polymyxin B are expressed in μg/mL, and are compared
to reference clinical breakpoints designated by CLSI. MICs of D-CONGA and D-CONGA-Q7
are expressed in μM and do not have standard reference breakpoints, as they are investigational.
Bold values indicate antibiotic cross-resistance.

| | | | | | | D- |
|---|---|---|---|---|---|---|
| | | | | Polymyxin | D- | CONGA- |
| Tx\Tested | AZ | CIP | COL | B | CONGA | Q7 |
| MPAO1-ΔmutS | 107 (13) | 4 (4) | — | 2 (1) | 5 (0.33) | 7 (0.42) |
| AZ | | | | | | |
| MPAO1-ΔmutS | 32 (4) | 8 (12) | — | 2 (1) | 8 (1) | 8 (1) |
| Chl | | | | | | |
| MPAO1-ΔmutS | 2 (0.33) | 0.5 (0.6) | 53 (20) | 27 (8) | 24 (3) | 19 (1.75) |
| COL | | | | | | |
| MPAO1-ΔmutS | 16 (1.5) | 0.5 (0.86) | — | 3 (1.33) | 16 (2) | 21 (2.67) |
| D-CONGA | | | | | | |
| MPAO1-ΔmutS | 4 (0.375) | 0.5 (0.67) | — | 4 (2) | 27 (3.33) | 27 (3.33) |
| D-CONGA-Q7 | | | | | | |
| MPAO1-ΔmutS | 8 (1) | 0.5 (0.75) | 2 (1) | 2 (1) | 8 (1) | 8 (1) |
| No Antibiotic | | | | | | |
| MPAO1-ΔmutL | 32 (2) | — | — | 0.5 (0.25) | — | — |
| Chl + Str | | | | | | |
| MPAO1-ΔmutL | 11 (0.8) | — | — | 2 (0.6) | — | — |
| No Antibiotic | | | | | | |
| MPAO1 | 8 (8) | 0.25 (0.3) | — | 3 (1.3) | 8 (1.2) | 8 (1.2) |
| AZ | | | | | | |
| MPAO1 | 2 (1) | 1 (1) | — | 2 (1) | — | — |
| Chl | | | | | | |
| MPAO1 | 2 (0.375) | 2 (2) | 4 (2) | 2 (1) | 8 (1) | 8 (1) |
| COL | | | | | | |
| MPAO1 | 7 (1.67) | 1 (3) | — | 4 (2.4) | 8 (1) | 8 (1) |
| D-CONGA | | | | | | |
| MPAO1 | 7 (1.67) | 0.75 (1.8) | — | 2 (1) | 11 (1.4) | 11 (1.33) |
| D-CONGA-Q7 | | | | | | |
| MPAO1 | 3 (1.25) | 2 (1) | — | — | — | — |
| Chl + Str | | | | | | |
| MPAO1 | 3 (1.67) | 2 (4.8) | 2 (1) | 1 (1) | 8 (2) | 8 (1) |
| No Antibiotic | | | | | | |

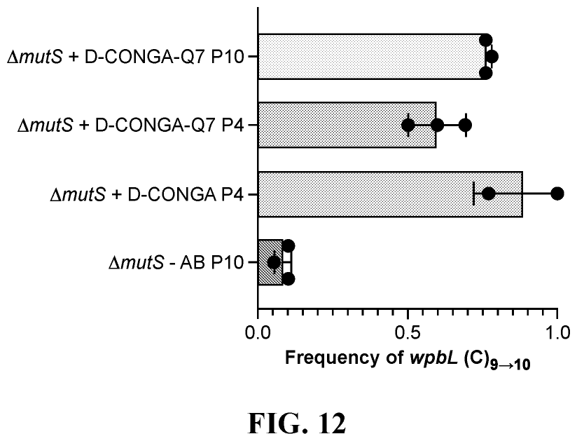

To elucidate potential mechanisms of cross-resistance, all non-synonymous de novo mutations in emergent resistant clones were examined. Antibiotic-treated clones were enriched in mutations in multiple transcriptional repressors that regulate mex drug efflux operons (Table 5). Drug efflux pumps are well-characterized transmembrane transport systems in P. aeruginosa and other bacteria that mediate antibiotic resistance via increased drug efflux (24, 25). Mutations in peptide-treated clones showed signatures of parallel evolution in pmrB, wbpL, and opr86, all associated with outer membrane modifications (Table 6) (26-31). A frameshift mutation was observed in wbpL present in low allelic frequency among all MPAO1-ΔmutS clones that was positively enriched only in peptide-treated clones (FIG. 12 and Table 7).

TABLE 5
Candidate resistance-conferring mutations of evolved MPAO1-ΔmutS
induced by aztreonam or chloramphenicol treatment. All identified de novo mutations with
high resistance-conferring probability were in transcriptional regulators of Mex efflux pump
operons.

| | | | | In MMR- | Gene | |
|---|---|---|---|---|---|---|
| | | | | deficiency | product | Predicted |
| Strain | Tx | Gene | Mutation | spectrum? | function | effect |
| MPAO1- | AZ | mexI | A806V | Yes | RND efflux | Altered |
| ΔmutS | | | GCG→GTG | | transporter | MexGHI- |
| | | | | | | OpmD |
| | | | | | | function |
| MPAO1- | AZ | mexD | N85S | Yes | RND efflux | Altered |
| ΔmutS | | | AAC→AGC | | transporter | MexGHI- |
| | | | | | | OpmD |
| | | | | | | function |
| MPAO1- | AZ | nalD | W205* | Yes | Secondary | Derepression |
| ΔmutS | | | TGG→TGA | | repressor of | of MexAB- |
| | | | | | MexAB- | OprM |
| | | | | | OprM | |
| MPAO1- | Chl | nalC | V40M | Yes | Repressor of | Derepression |
| ΔmutS | | | GTG→ATG | | ArmR | of MexAB- |
| | | | | | | OprM |
| MPAO1- | Chl | — | (G)_{5→4} | Yes | — | — |
| ΔmutS | | | 147/642 nt | | | |
| MPAO1- | Chl | mexT | T311I | Yes | Global LysR | Induction of |
| ΔmutS | | | ACC→ATC | | transcriptional | MexEF- |
| | | | | | regulator | OprN |
| MPAO1- | Chl | — | F314L | Yes | — | — |
| ΔmutS | | | TTC→CTC | | | |
| MPAO1 | AZ | nalD | T158P | No | Secondary | Derepression |
| | | | ACC→CCC | | repressor of | of MexAB- |
| | | | | | MexAB- | OprM |
| | | | | | OprM | |

TABLE 6
Chromosomal mutations in MMR-deficiency spectrum revealed novel gene
targets for mechanism of resistance of D-CONGA and D-CONGA-Q7. Three genes (pmrB,
opr86, and wbpL) were mutated in parallel among independently evolved lineages of MPAO1-
ΔmutS that had elevated MICs to D-CONGA and D-CONGA-Q7. pmrB-V361M was present
at a frequency of 56%.

| | | | | In MMR- | | |
|---|---|---|---|---|---|---|
| | | | | deficiency | Gene product | Predicted |
| Strain | Tx | Gene | Mutation | spectrum? | function | effect |
| MPAO1- | D- | wbpL | (C)_{7→8} | Yes | Glycosyl- | Frameshift |
| ΔmutS | CONGA | | 140/1020 nt | | transferase | and loss-of- |
| | | | | | WbpL | function; |
| | | | | | | possible O- |
| | | | | | | antigen mod. |
| MPAO1- | D- | — | (C)_{9→10} | Yes | — | — |
| ΔmutS | CONGA | | 221/1020 nt | | | |
| MPAO1- | D- | opr86 | D603G | Yes | Outer | AMP |
| ΔmutS | CONGA | | GAC→GGC | | membrane | resistance |
| | | | | | protein Opr86 | (polymyxin |
| | | | | | | B and COL) |
| MPAO1- | D- | — | Q433R | Yes | — | — |
| ΔmutS | CONGA | | CAG→CGG | | | |
| MPAO1- | D- | pmrB | A248T | Yes | Two- | Reduction of |
| ΔmutS | CONGA | | GCC→ACC | | component | net charge of |
| | | | | | regulator | LPS by |
| | | | | | system signal | Ara4N |
| | | | | | sensor kinase | incorporation |
| | | | | | | in lipid A |
| MPAO1- | D- | opr86 | Q433R | Yes | Outer | AMP |
| ΔmutS | CONGA- | | CAG→CGG | | membrane | resistance |
| | Q7 | | | | protein Opr86 | (polymyxin |
| | | | | | | B and COL) |
| MPAO1- | D- | — | D655G | Yes | — | — |
| ΔmutS | CONGA- | | GAC→GGC | | | |
| | Q7 | | | | | |
| MPAO1- | D- | pmrB | G188S | Yes | Two- | Reduction of |
| ΔmutS | CONGA- | | GGC→AGC | | component | net charge of |
| | Q7 | | | | regulator | LPS by |
| | | | | | system signal | Ara4N |
| | | | | | sensor kinase | incorporation |
| | | | | | | in lipid A |
| MPAO1- | D- | — | V361M | Yes | — | — |
| ΔmutS | CONGA- | | GTG→ATG | | | |
| | Q7 | | | | | |

TABLE 7
Allelic frequency of variant wbpL (C)_{9→10} (221/1020 nt) in the
sequenced populations of evolved MPAO1-ΔmutS lineages.

| Lineage | Resistance to | Allelic frequency |
|---|---|---|
| ΔmutS-3-CONGA-P4 | D-CONGA, D-CONGA-Q7 | 77% |
| ΔmutS-1-Q7-P4 | D-CONGA, D-CONGA-Q7 | 50% |
| ΔmutS-1b-Q7-P4 | D-CONGA, D-CONGA-Q7 | 60% |
| ΔmutS-2b-Q7-P4 | D-CONGA, D-CONGA-Q7 | 69% |
| ΔmutS-1-Q7-P10 | D-CONGA, D-CONGA-Q7, | 78% |
| | polymyxin B | |
| ΔmutS-1b-Q7-P10 | D-CONGA, D-CONGA-Q7, | 76% |
| | polymyxin B | |
| ΔmutS-2b-Q7-P10 | D-CONGA, D-CONGA-Q7, | 76% |
| | polymyxin B | |
| ΔmutS-1-noAB-P10 | None | 10% |
| ΔmutS-2-noAB-P10 | None | 10% |
| ΔmutS-3-noAB-P10 | None | 5% |

To validate membrane-modifications as the mechanism of resistance to peptides D-CONGA and D-CONGA-Q7, lipid A was purified from D-CONGA- and D-CONGA-Q7-treated clones and matrix-assisted laser desorption ionization mass spectrometry (MALDI-MS) was performed. D-CONGA-treatment selected against the presence of penta-acylated lipid A (m z 1446) and secondary palmitoyl-modified lipid A (m z 1684), suggesting downregulation of PagL and PagP promotes D-CONGA resistance (FIG. 2C) (32, 33). D-CONGA-Q7-treated clones predominately showed presence of penta-acylated lipid A (m z 1446) and secondary palmitoyl-modified lipid A (m z 1684), suggesting upregulation of PagL and PagP promote D-CONGA-Q7 resistance. Two clones showed 4-amino-4-deoxy-L-arabinose (Ara4N) incorporation (m z 1815), which confers antimicrobial peptide resistance (FIG. 2C, right) (29). These spectra show marked differences in selection for lipid A structure between D-CONGA and D-CONGA-Q7, and support membrane modifications as the mechanism of resistance for peptides.

Whole genome sequencing and de novo mutation analysis showed parallel evolution of nonsynonymous mutations in efflux pumps for aztreonam- and chloramphenicol-treated lineages and in membrane-modifying genes for peptide-treated lineages. Notably, no mutations in mex drug efflux operons were found in peptide-treated clones, and no mutations in membrane modification genes were found in antibiotic-treated clones (FIG. 2D). Our data thus far suggest that treatment of MMR-deficient P. aeruginosa drives cross-resistance through shared mechanisms of resistance, and that the mechanisms of resistance for antibiotic compounds and peptides are distinct and exclusive.

### Example 4: Rational Combination Therapy of MMR-Deficient P. aeruginosa Prevents Resistance Acquisition

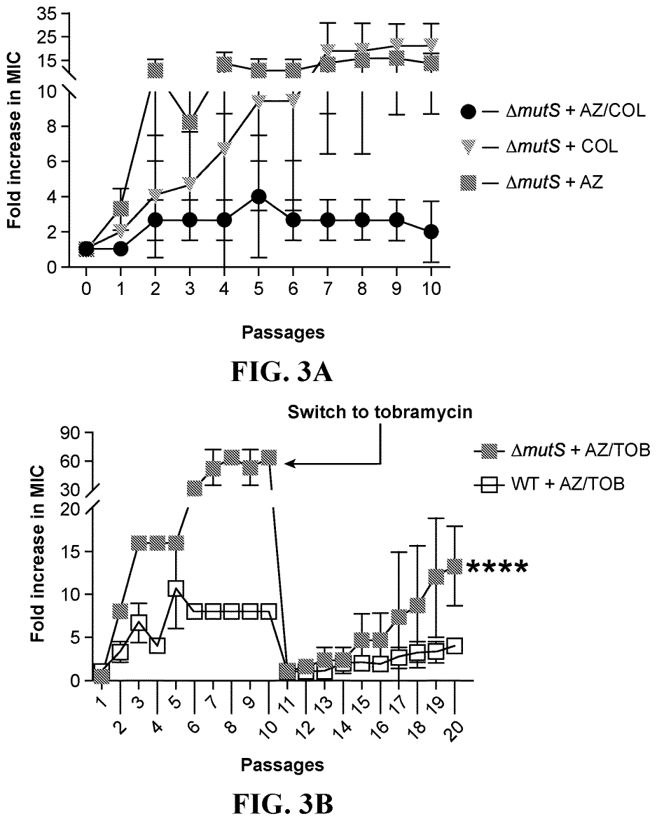

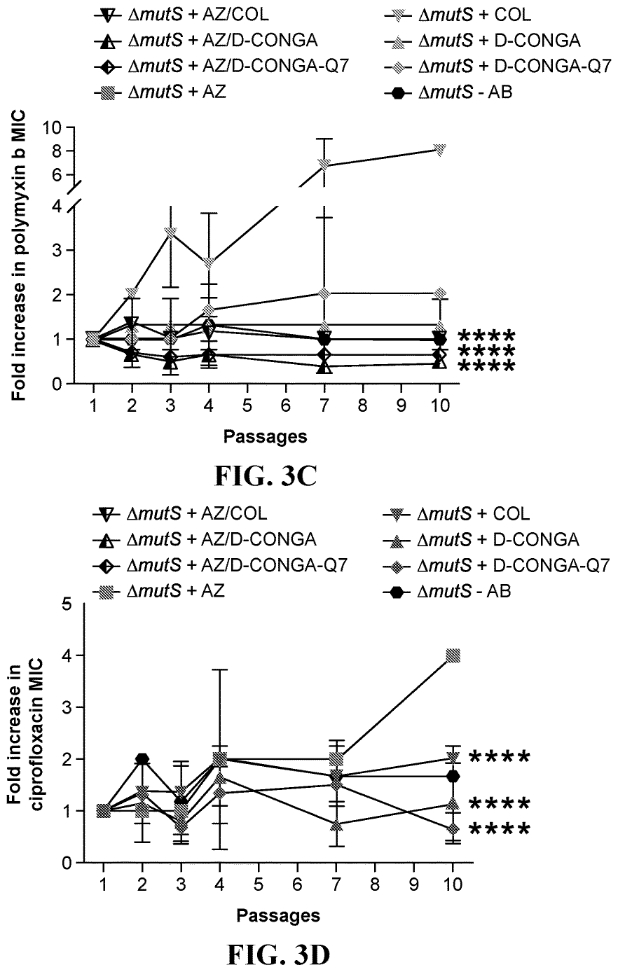

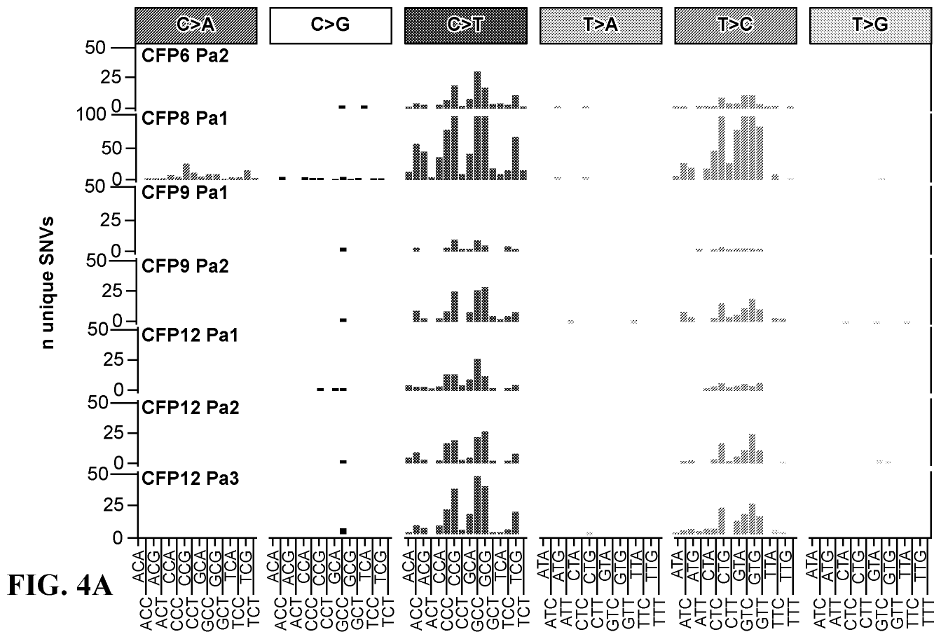

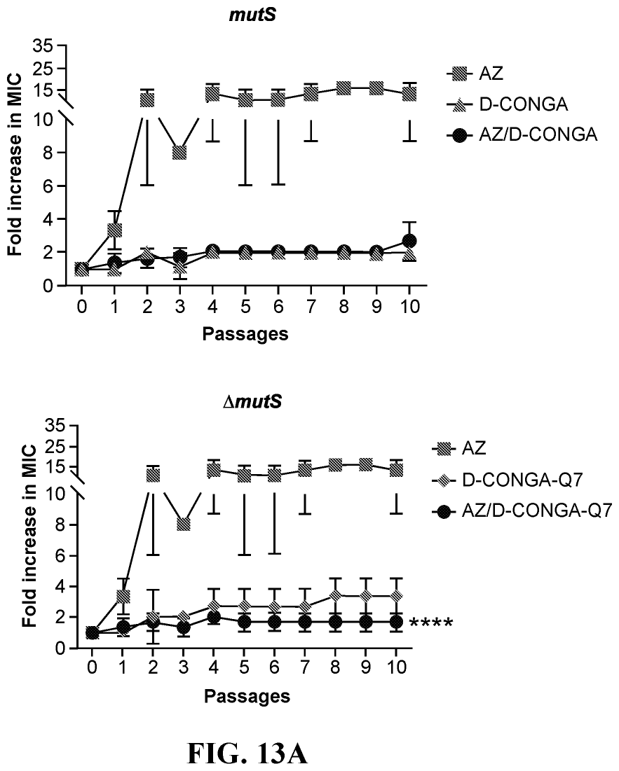

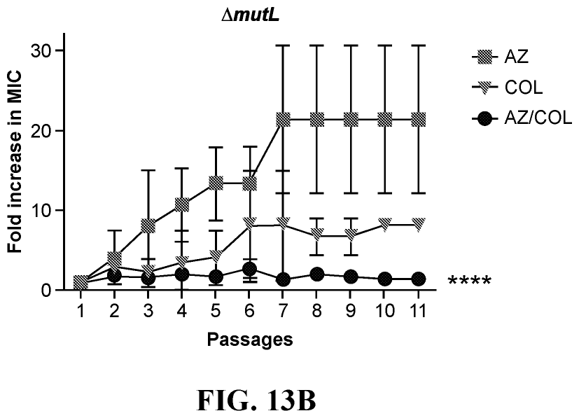

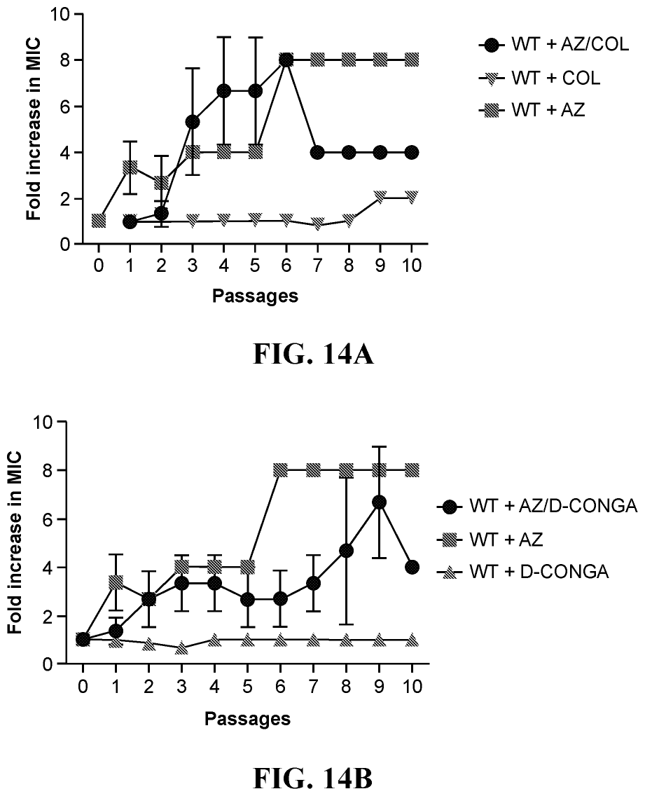

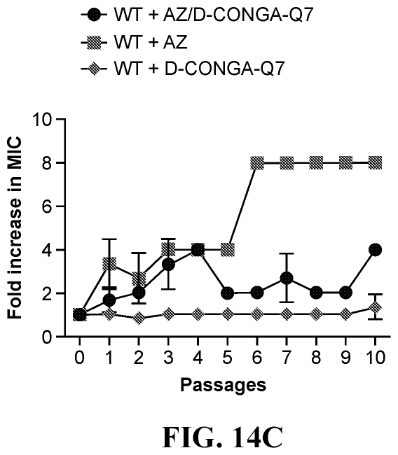

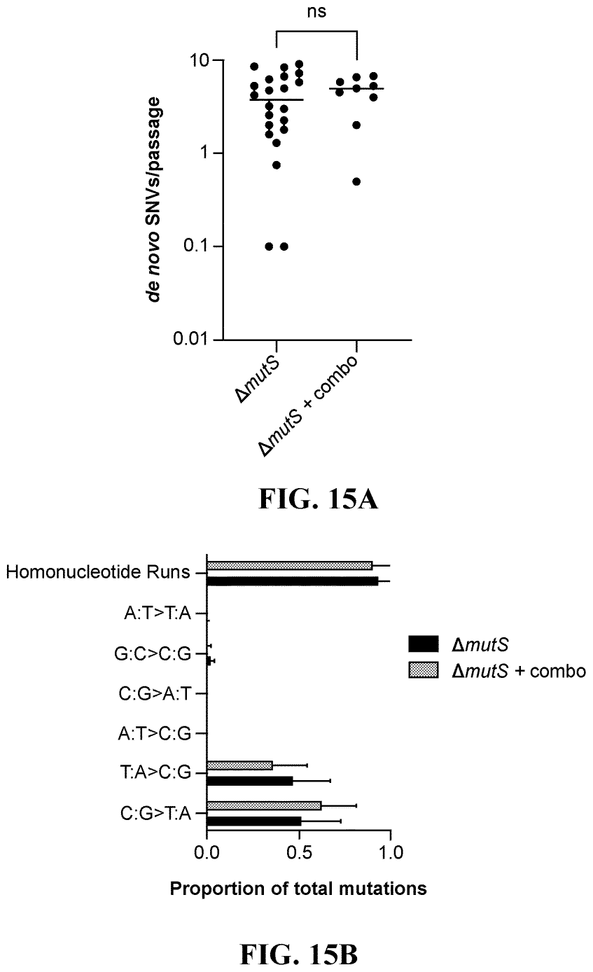

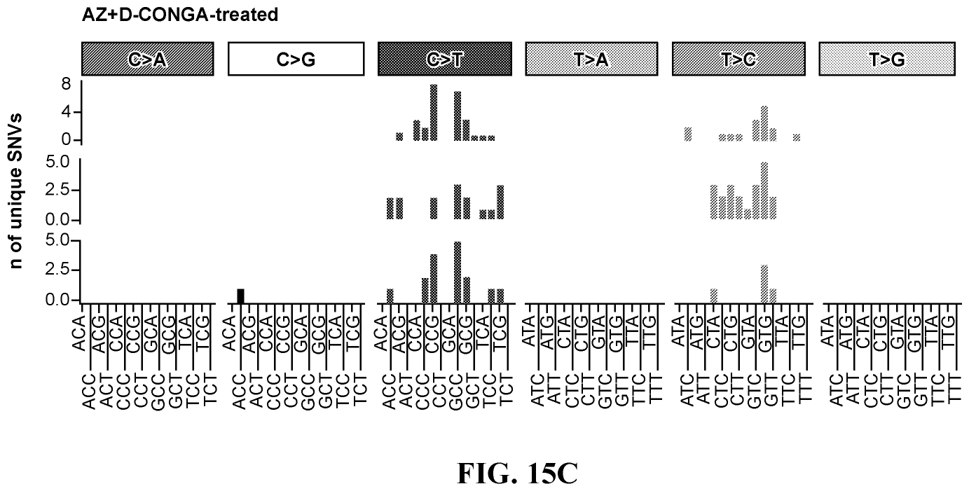

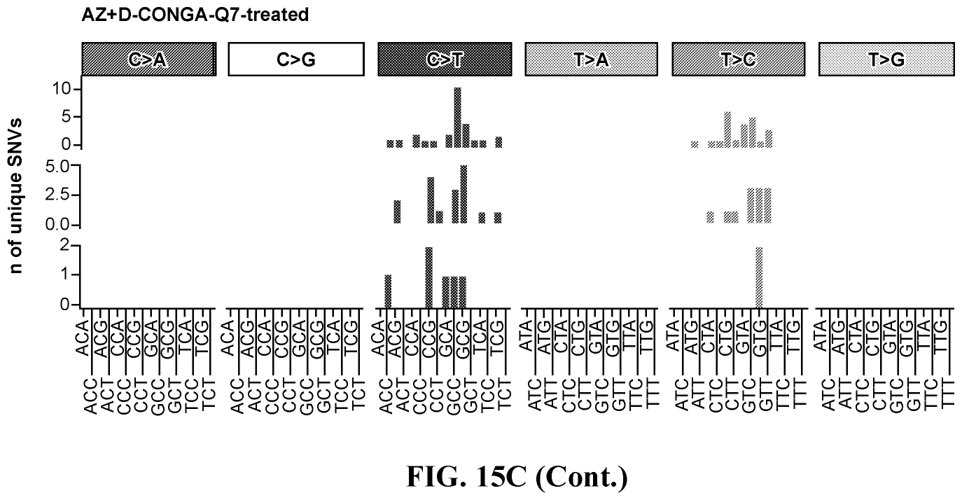

Based on the mutual exclusivity of resistance mutations to antibiotics and peptides, and without wishing to be bound by any particular theory, it was postulated that combining treatments that require distinct resistance mechanisms could potentially prevent MDR in MMR-deficient P. aeruginosa, perhaps due to excessive mutation burden required for two or more resistance pathways. To test this, MPAO1-ΔmutS was evolved in antibiotic+peptide combinations (aztreonam+colistin, aztreonam+D-CONGA, and aztreonam+D-CONGA-Q7) and measured resistance acquisition after repeated combination treatment compared to monotherapy. All antibiotic+peptide combination therapies significantly reduced resistance acquisition compared to monotherapies (FIG. 3A and FIGS. 13A-13B). In vitro adaptive evolution of MPAO1-ΔmutL in aztreonam+colistin also showed reduced resistance acquisition, suggesting that efficacy of combination therapy is not dependent on MMR-deficiency genotype (FIG. 13C). Antibiotic+peptide combination therapies showed no significant effect on resistance acquisition in MMR-proficient MPAO1 (FIGS. 14A-14C). WGS of midpoint and endpoint clones showed that combination therapy had no effect on mutation rate (FIG. 15A) or mutation spectra (FIGS. 15B-15C), indicating combination therapy was effective despite continued mutagenesis from MMR-deficiency.

A common treatment approach for pwCF chronically infected with P. aeruginosa is cycled continuous inhaled antibiotic therapy (19, 34, 35). To emulate this in vitro, MPAO1-ΔmutS was treated with aztreonam and then evolved the adapted clones in tobramycin and determined resistance acquisition. Aztreonam cycled with tobramycin induced significant resistance in MPAO1-ΔmutS. MPAO1 also acquired resistance to aztreonam cycled with tobramycin, albeit at a much slower rate and magnitude (FIG. 3B).

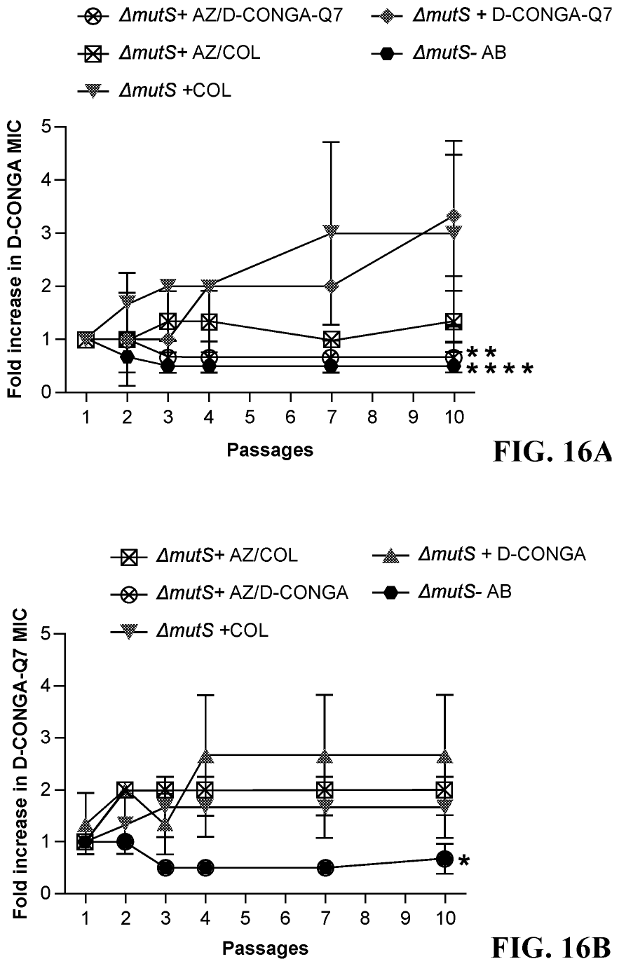

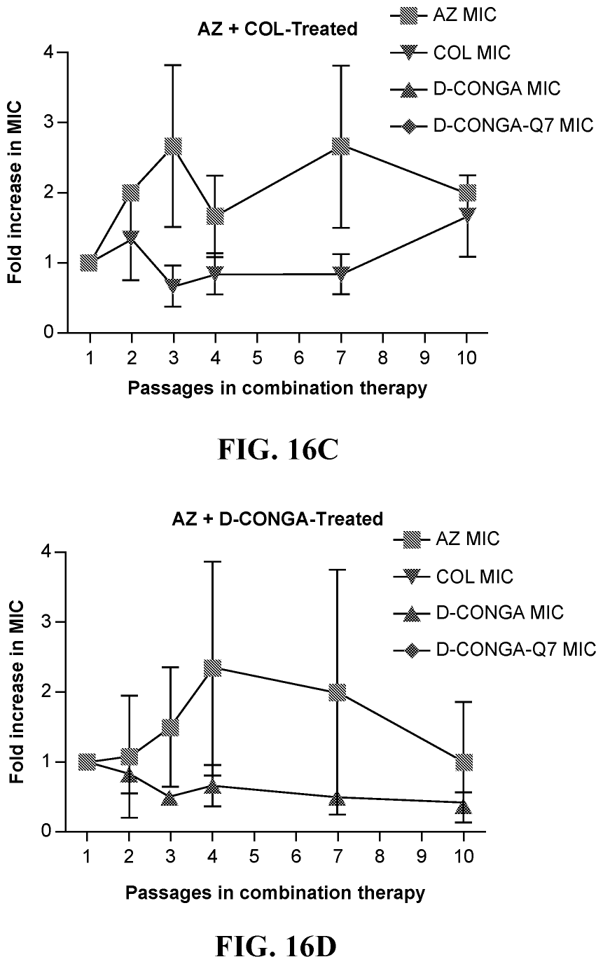

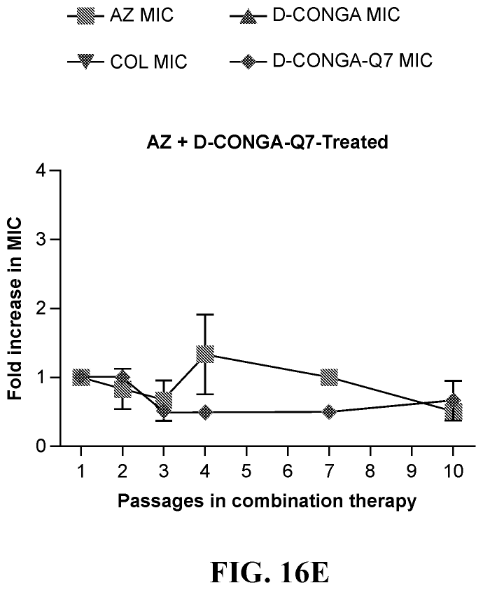

To determine if antibiotic+peptide combination therapy could eliminate treatment-induced MDR acquisition in MMR-deficient P. aeruginosa (FIG. 2), combination-treated evolved clones were evaluated against the same panel of antibiotics for cross-resistance acquisition. Antibiotic+peptide combination therapy treatment of MPAO1-ΔmutS effectively eliminated cross-resistance to polymyxin B, D-CONGA, and D-CONGA-Q7 previously observed with peptide monotherapy (FIG. 3C and FIGS. 16A-16B). Combination therapy of MPAO1-ΔmutS also eliminated cross-resistance acquisition to ciprofloxacin, previously observed with antibiotic monotherapies (FIG. 3D). Combination-treated clones did not show any significant elevation in MICs against relevant monotherapies (FIG. 16C-16E). A summary of all MIC values is in Table 8. Without wishing to be bound to any particular theory, the data suggests that rationally combining therapies requiring exclusive mechanisms of resistance prevents multidrug resistance acquisition in MMR-deficient P. aeruginosa.

TABLE 8
Summary of observed MICs across drug classes following evolution of
MPAO1-ΔmutS and MPAO1 with combination treatments. Average MICs (independent
biological triplicates) of respective drug at end points following evolution of MPAO1-ΔmutS
or MPAO1 in aztreonam + colistin, aztreonam + D-CONGA, aztreonam + D-CONGA-Q7, or
no antibiotic treatment. Values are expressed as absolute MIC (fold-change relative to starting
MIC before treatment), and MICs of combination treatments are expressed as aztreonam
MIC/AMP MIC. MICs of aztreonam, ciprofloxacin, colistin, and polymyxin B are expressed
in μg/mL, and are compared to reference clinical breakpoints designated by CLSI. MICs of D-
CONGA and D-CONGA-Q7 are expressed in μM and do not have standard reference
breakpoints, as they are investigational.

| | | | | | | | D- |
|---|---|---|---|---|---|---|---|
| | | | | | Polymyxin | D- | CONGA- |
| Tx\Tested | Tx | AZ | CIP | COL | B | CONGA | Q7 |
| MPAO1- | 11/1 (1.7) | 16 (2) | 2 (2) | 3 (1.67) | 2 (1) | 7 (1.3) | 16 (2) |
| ΔmutS | | | | | | | |
| AZ + COL | | | | | | | |
| MPAO1- | 11/5 (2.7) | 18 (1) | 0.8 | — | 2 (1) | 7 (0.43) | 11 (0.67) |
| ΔmutS | | | (1.2) | | | | |
| AZ + D- | | | | | | | |
| CONGA | | | | | | | |
| MPAO1- | 7/3 (1.7) | 16 (1) | 0.7 | — | 2 (1) | 8 (0.67) | 11 (0.67) |
| ΔmutS | | | (0.7) | | | | |
| AZ + D- | | | | | | | |
| CONGA-Q7 | | | | | | | |
| MPAO1- | — | 5 | 0.5 | 2 (1) | 2 (1) | 8 (1) | 11 (1.38) |
| ΔmutS | | (0.67) | (0.5) | | | | |
| No Antibiotic | | | | | | | |
| MPAO1 | 8/1 (4) | 19 (9) | 2 (2) | 5 (2.67) | 2 (1) | 8 (1) | 8 (1) |
| AZ + COL | | | | | | | |
| MPAO1 | 4/2 (4) | 11 | 0.7 | — | 2 (1) | 8 (1.2) | 8 (1.2) |
| AZ + D- | | (2.67) | (0.84) | | | | |
| CONGA | | | | | | | |
| MPAO1 | 4/2 (2) | 4 (1) | 0.8 (1) | — | 2 (1) | 8 (1.2) | 8 (1.2) |
| AZ + D- | | | | | | | |
| CONGA-Q7 | | | | | | | |
| MPAO1 | — | 4 (1) | 0.8 (1) | 2 (1) | 2 (1) | 8 (1) | 8 (1) |
| No Antibiotic | | | | | | | |

It is anticipated that combination therapy with cephalosporins (e.g., ceftazidime, cefiderocol) or carbapenems (e.g. meropenem, imipenem) and an antipseudomonal cationic peptide (e.g., colistin, polymyxin B) will also be effective in treating a hypermutator Pseudomonas infection.

### Example 5: Mutational Signature Analysis Predicts MMR-Deficiency and MDR in P. aeruginosa Isolates from pwCF

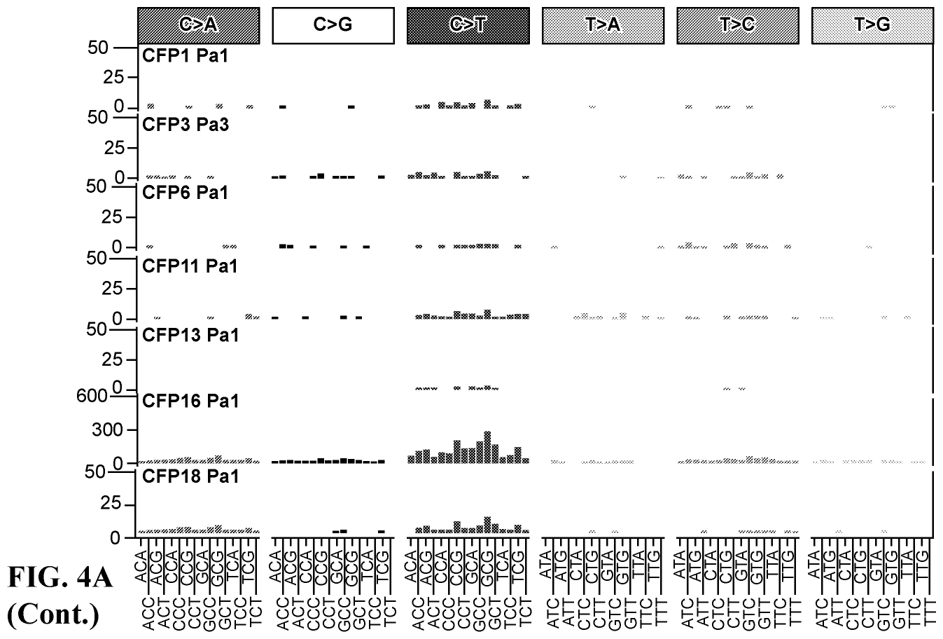

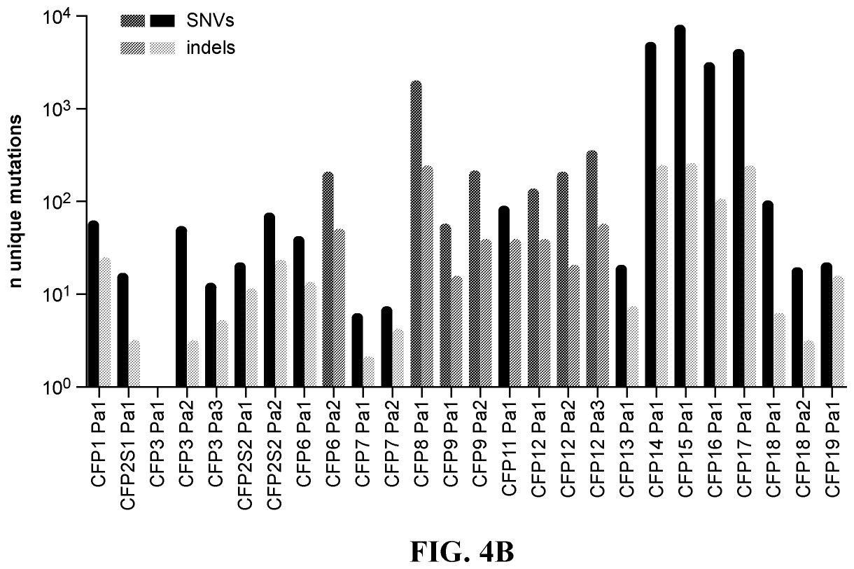

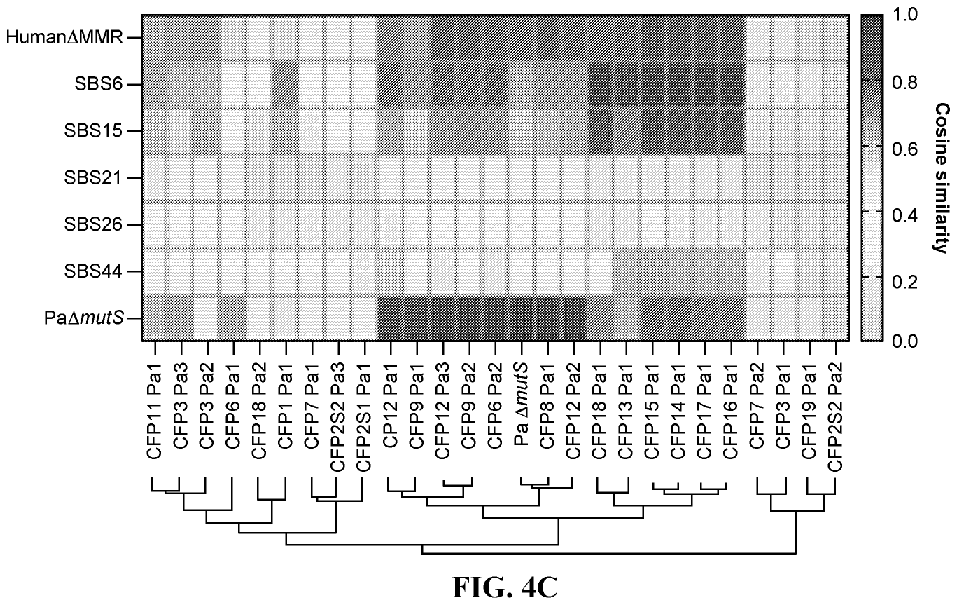

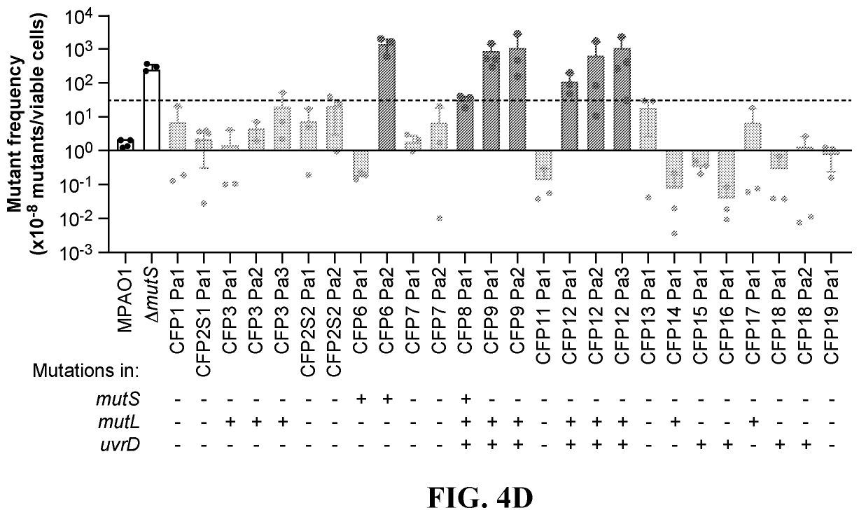

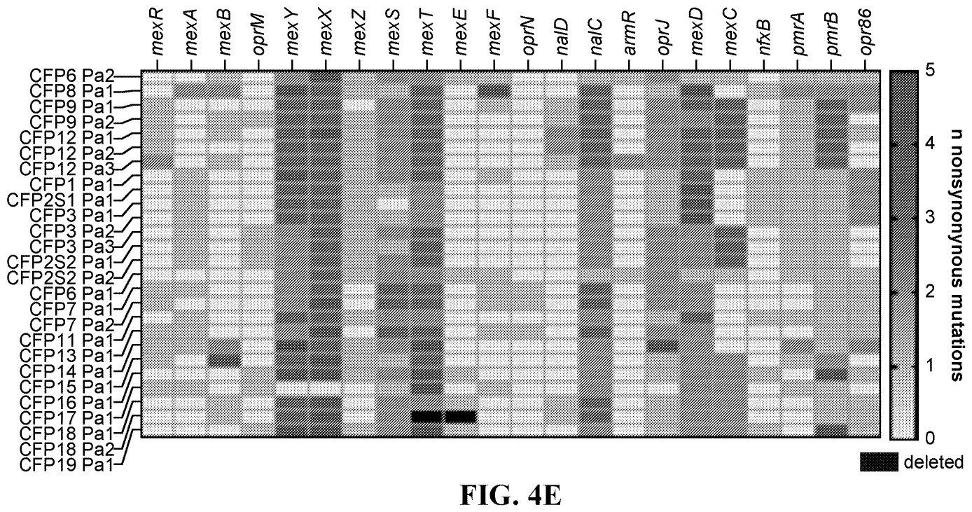

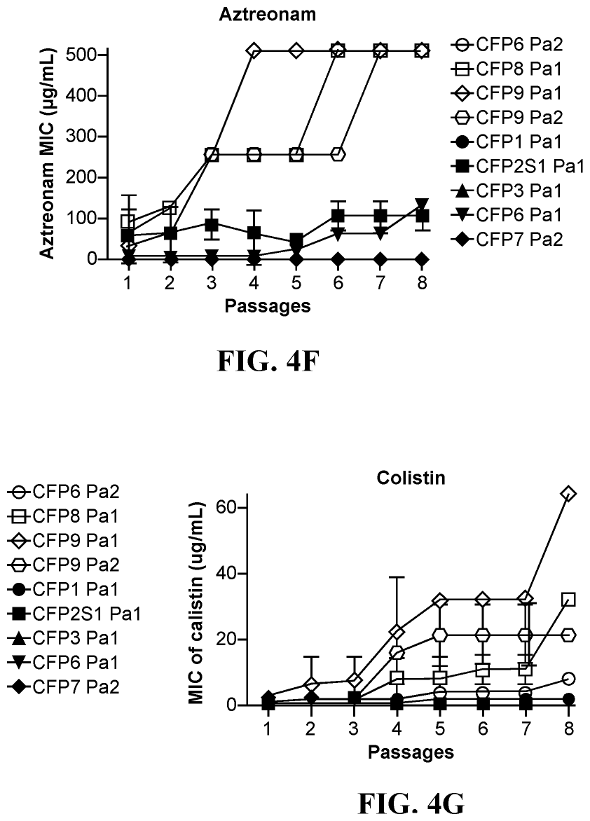

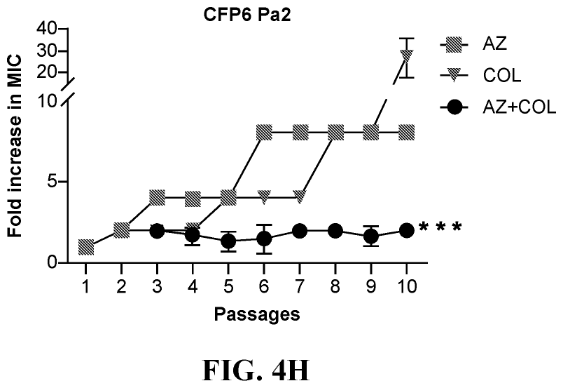

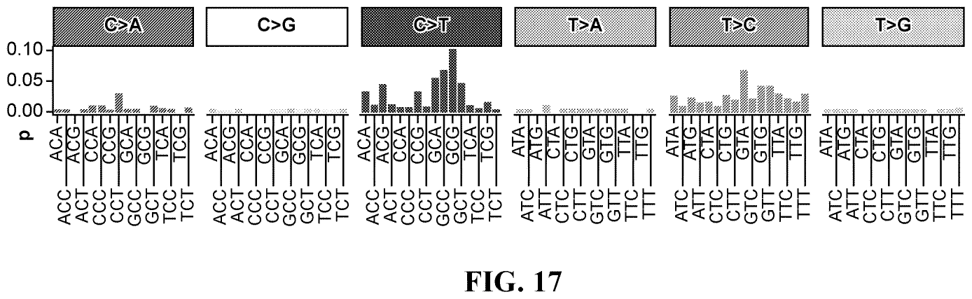

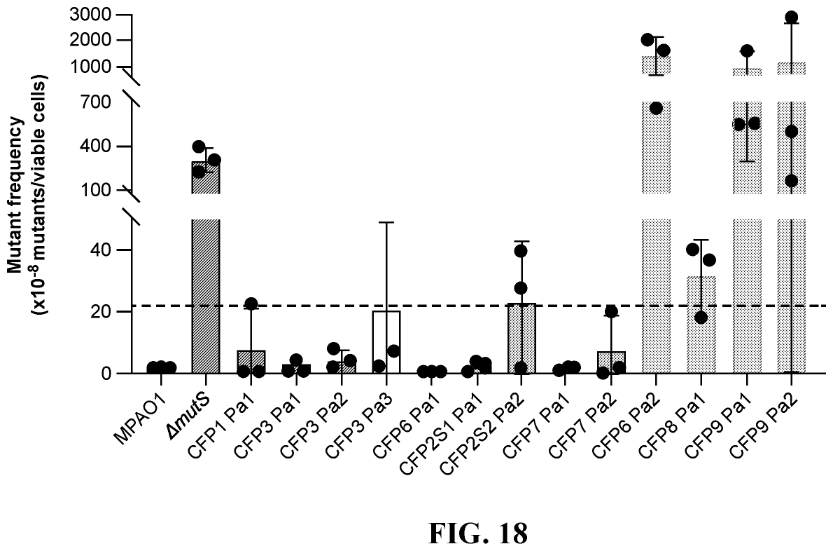

Next it was determined if the MMR-status of P. aeruginosa isolated from pwCF could be predicted from WGS and mutational signature analyses. Eight sputum samples from seven pwCF (two longitudinally from one patient) was collected, resulting in the isolation of 14 strains of P. aeruginosa. Mutation spectra from four samples, CFP6_Pa2, CFP8_Pa1, CFP9_Pa1, and CFP9_Pa2, showed high mutation burdens and mutational signatures consistent with MMR-deficiency and hypermutator phenotype (FIG. 4A). Unique SNVs and indels per isolate following deduplication of variants across all samples, with predicted MMR-deficient isolates (FIG. 4B). Quantitative assessment via cosine similarity showed that mutation signatures from these isolates were most similar to those from mutS-deficient P. aeruginosa laboratory strains (FIG. 4C). The P. aeruginosa MMR-deficient mutational signature and spectra of all subject isolates was also compared to COSMIC SBS signatures associated with MMR-deficiency in human tumors (FIG. 17) and relatively high similarity was found despite notable shifts in trinucleotide context preferences (FIG. 4C). Additionally, different subject isolates clustered based on decreasing degrees of similarity to the P. aeruginosa MMR-deficient mutational signature which may indicate an intermediate MMR-deficiency state. The four clinical isolates that possessed a mutational signature most similar to mutS-deficient P. aeruginosa were also found to be hypermutators using the rifampicin reversion assay, confirming the potential diagnostic utility of mutational signature analysis (FIG. 18).

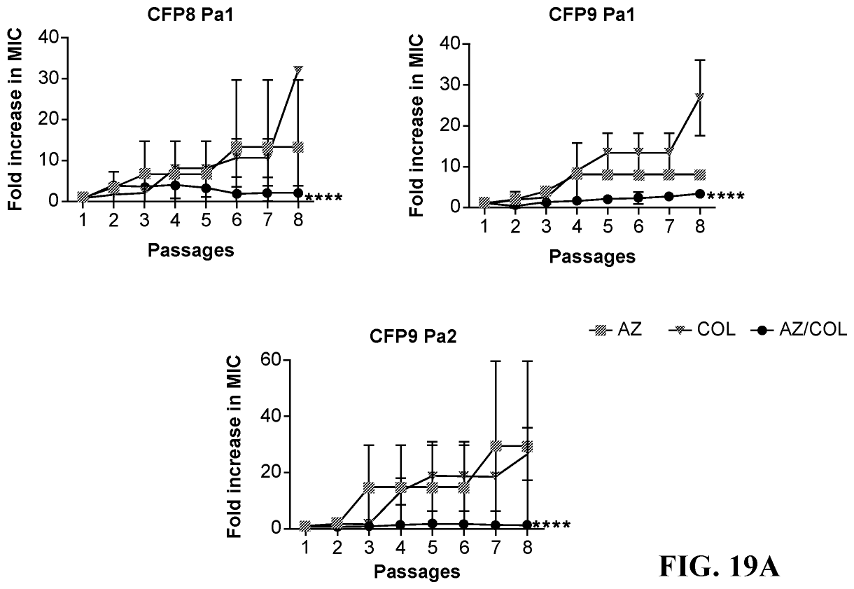

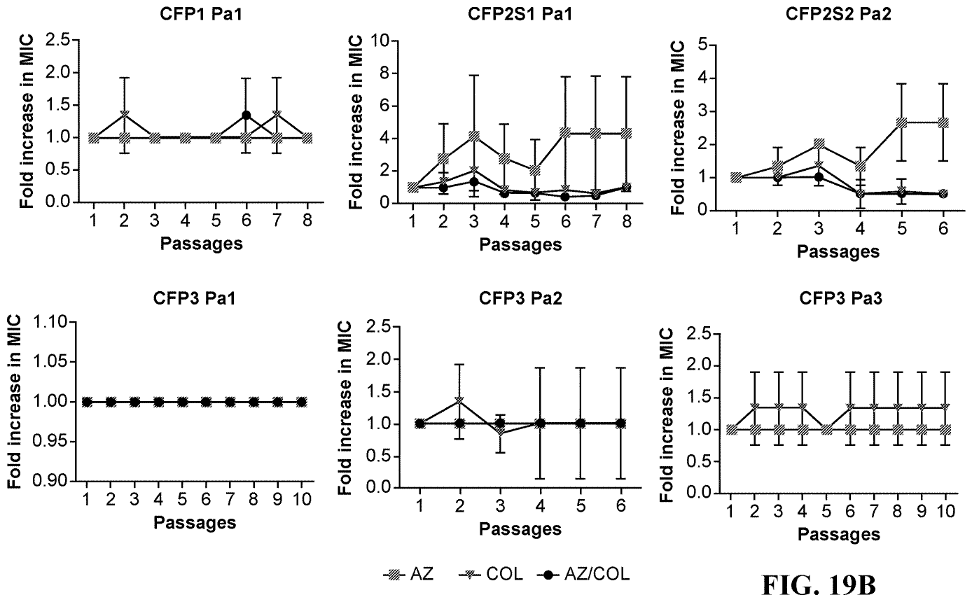

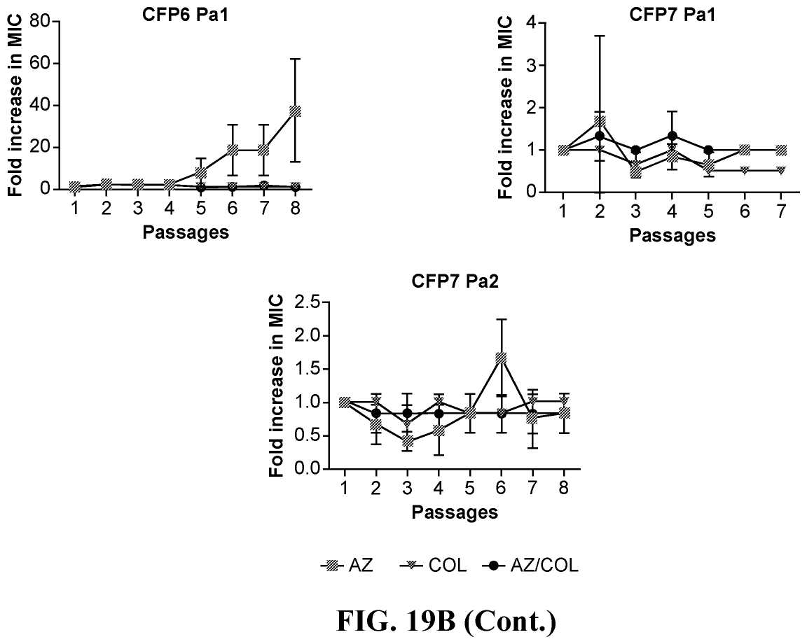

Next, resistance acquisition and efficacy of combination therapy in the predicted MMR-deficient clinical isolates was measured in vitro. All strains were evolved in aztreonam alone, colistin alone, and combined aztreonam+colistin and measured resistance acquisition. Three predicted MMR-deficient isolates, CFP6_Pa2, CFP8_Pa1, and CFP9_Pa1, demonstrated MICs to aztreonam above defined clinical breakpoints (determined by Clinical and Laboratory Standards Institute (CLSI)) at the start of in vitro adaptive evolution. All three isolates acquired further resistance to aztreonam alone and rapidly acquired resistance to colistin (FIGS. 4F-4G and FIG. 19A). CFP9_Pa2 was heterogeneously susceptible to both monotherapies, but all clones rapidly acquired resistance similar to MMR-deficient P. aeruginosa laboratory strains (FIGS. 4F-4G). CFP6_Pa1, with a seemingly intermediate MMR-deficient state (FIGS. 4F-4G), acquired resistance to aztreonam but at a slower rate than predicted MMR-deficient isolates (FIGS. 4F-4G). Aztreonam+colistin combination therapy prevented resistance acquisition in all MMR-deficient clinical isolates, even in those with preexisting aztreonam-resistance (FIG. 4H and FIG. 19A). Predicted MMR-proficient clinical isolates did not develop significant resistance acquisition, and on these combination therapy had no effect on resistance acquisition (FIGS. 4F-4G and FIG. 19B). Together, these results indicate that mutational signature analysis coupled with rational combination therapy can prevent the emergence of hypermutator-induced MDR in P. aeruginosa clinical isolates in vitro.

Without wishing to be bound to any particular theory, results suggest that MMR-deficiency, while producing a hallmark mutational signature, drives rapid MDR acquisition but can be targeted with rational combination therapy. As such, the identification of the MMR-deficiency-associated mutational signature would be a predictor of rapid drug resistance acquisition and could potentially guide targeted treatment with combination therapy.

26 isolates of P. aeruginosa from 16 pwCF were collected and subsequently WGS was performed and plotted trinucleotide mutation spectra of all isolates to predict their MMR-status. Trinucleotide mutation spectra from seven patient isolates showed C>T enrichment in NCC and NCG and diminishment in NCT and T>C enrichment in CTN and GTN consistent with PaΔmutS (FIG. 4A). These seven isolates had elevated levels of unique single nucleotide variants (SNVs) and indels, especially comparing CFP6 Pa2, whose spectrum resembles PaΔmutS, with CFP6 Pa1 collected from the same patient at the same time, whose spectrum does not and therefore serves as a ‘matched control’ (FIG. 4B). Quantitative assessment via cosine similarity showed that spectra from these isolates were most similar to PaΔmutS (FIG. 4C). From cosine similarity analysis and clustering results, all isolates with a cosine similarity with PaΔmutS above 0.78 were predicted to be MMR-deficient, and all below 0.78 as WT, resulting in 7 predicted MMR-deficient isolates and 19 WT (27% isolates MMR-deficient). These six isolates show C>T enrichment but were lacking the dramatic C>T diminishment in NCT contexts and T>C enrichment, suggesting these are key distinguishing factors for prediction of MMR-status.

Predictions of MMR-status were functionally validated by assessing for hypermutator phenotype via rifampicin reversion frequency (FIG. 4D). Additionally, all predicted MMR-deficient isolates had a nonsynonymous mutation in mutS, mutL, or uvrD. However, many validated WT isolates also had mutations in an MMR gene, indicating that assessing for genotype is an inaccurate determinant of MMR-status (FIG. 4D). Subsequently, all isolates were assessed for mutations in mex efflux operons and membrane-modifying operons. MMR-deficient isolates appear to be slightly enriched in nalD mutations compared to WT isolates, which was also observed with aztreonam-treatment during in vitro adaptive evolution (FIG. 4E).

To assess if presence of the PaΔmutS mutational signature is a predictor of rapid drug resistance, resistance acquisition and the efficacy of combination therapy was measured in a sampling of predicted MMR-deficient and WT clinical isolates in vitro. All strains were evolved in aztreonam alone, colistin alone, and combined aztreonam+colistin and measured resistance acquisition. Three predicted MMR-deficient isolates, CFP6 Pa2, CFP8 Pa1, and CFP9 Pa1, demonstrated MICs to aztreonam above CLSI-defined clinical breakpoints at the start of in vitro adaptive evolution. All three isolates acquired further resistance to aztreonam alone and rapidly acquired resistance to colistin (FIGS. 4F-4G). CFP9 Pa2 was heterogeneously susceptible to both monotherapies, but all clones rapidly acquired resistance similar to MMR-deficient P. aeruginosa laboratory strains (FIGS. 4F-G). Aztreonam+colistin combination therapy prevented resistance acquisition in all predicted MMR-deficient clinical isolates, even in those with preexisting aztreonam-resistance (FIG. 4H and FIGS. 11B-11D). Predicted MMR-proficient clinical isolates either did not develop significant resistance acquisition or did so at a rate similar to WT MPAO1, and on these combination therapy had no effect on resistance acquisition (FIGS. 4F-4G and FIGS. 11E-11M). Together, these results indicate that mutational signature analysis coupled with rational combination therapy is a precision medicine approach that can prevent the emergence of MMR-deficiency-induced MDR in P. aeruginosa clinical isolates in vitro.

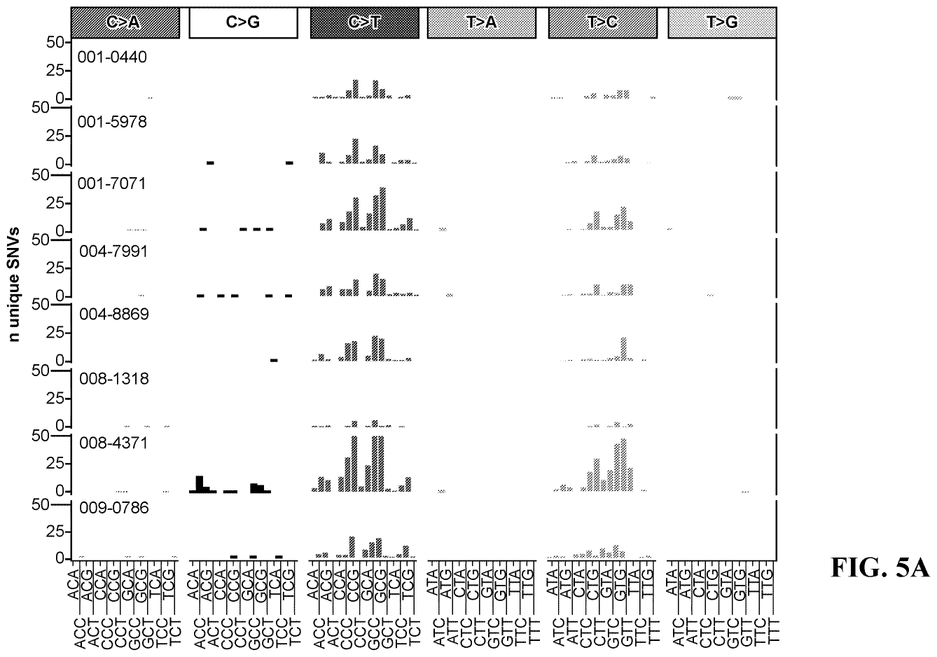

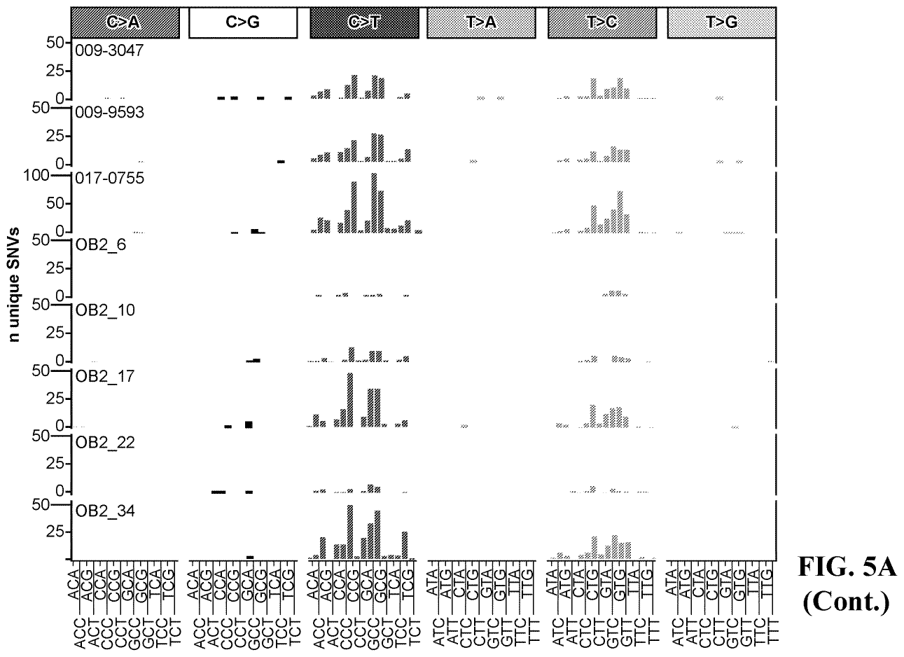

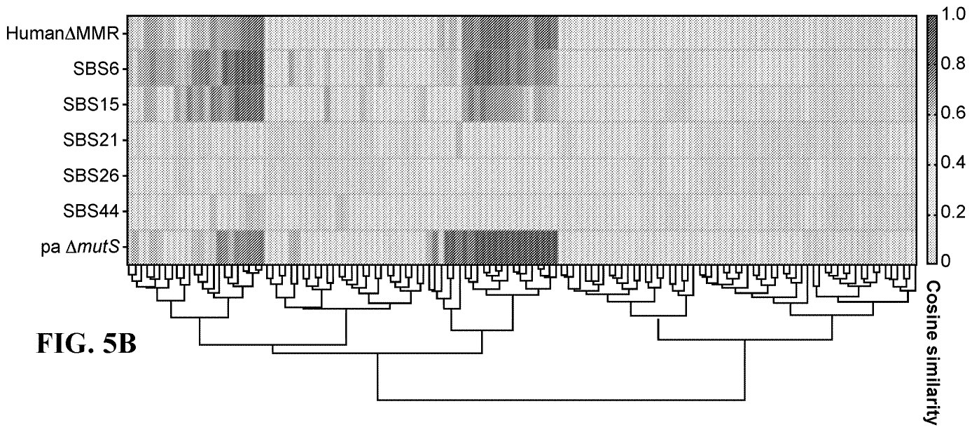

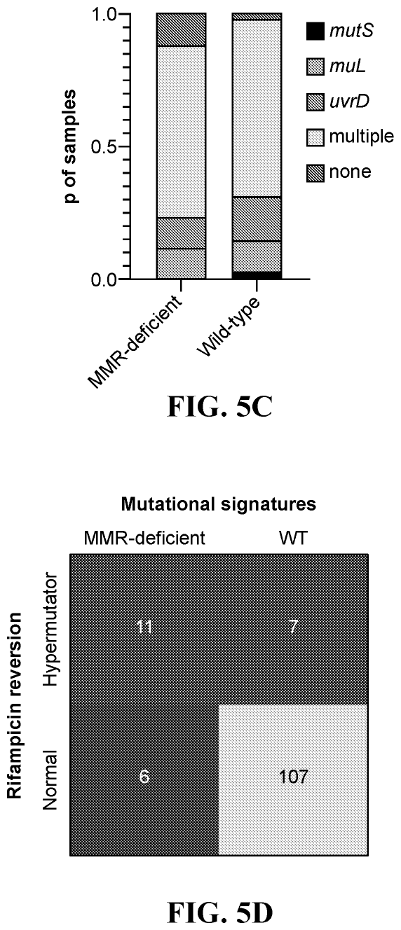

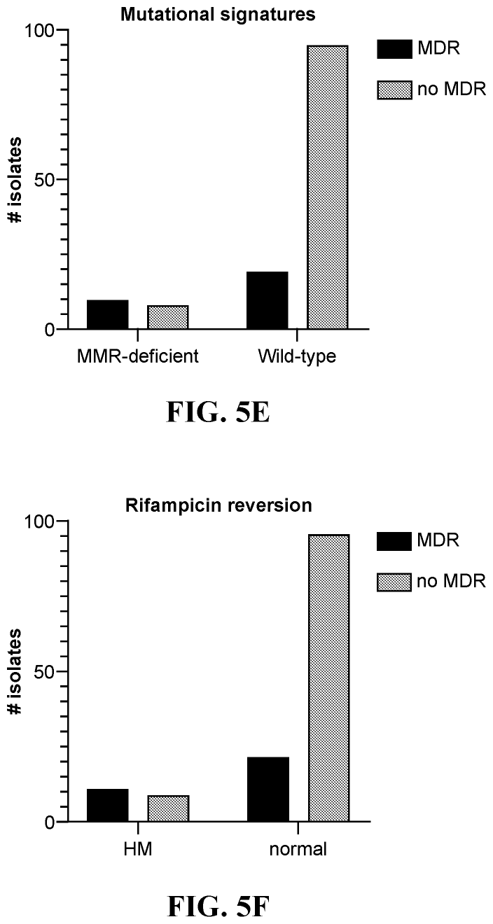

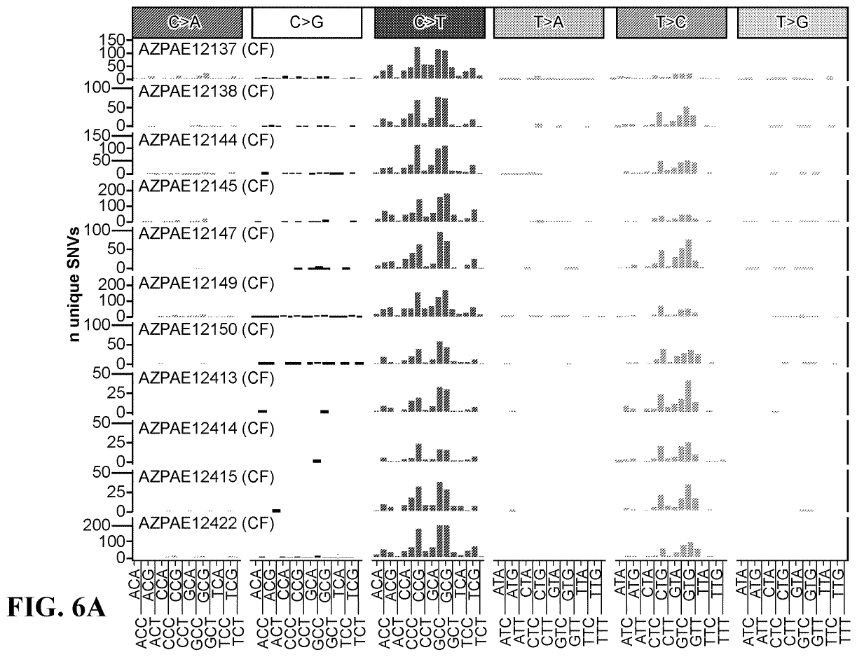

Publicly available WGS reads of 131 P. aeruginosa isolates from 50 pwCF in Spain was assessed. See Lopez-Causape et al. Trinucleotide mutation spectra from 17 isolates strongly resemble PaΔmutS and show enrichment and diminishment in key discussed C>T and T>C contexts (FIG. 5A). Cosine similarity analysis predicted these 17 isolates as MMR-deficient and the remaining 114 as WT (13% MMR-deficient). Data on clinical resistance and hypermutator phenotype also accompanied these isolates. 11 out of 17 predicted MMR-deficient isolates were reported as hypermutators via rifampicin reversion, whereas the other 6 were not. Additionally, 7 predicted WT isolates were reported to be hypermutators (FIG. 5D). Despite incomplete agreement with hypermutator phenotype, predicted MMR-deficient isolates show strong correlation (p=0.0021 via Fisher's exact) with MDR (defined by ‘R’ via EUCAST to ≥3 drugs) (FIG. 5E), as does the hypermutator phenotype as previously disclosed (FIG. 5F). Application of mutational signature analysis in clinical isolates of P. aeruginosa robustly, easily, and accurately screens for MMR-deficiency and identifies isolates prone to rapid MDR acquisition or having significantly higher rates of MDR.

### Example 6: Mutational Signature Analysis Reveals Presence of MMR-Deficiency in Other Contexts

The study of MMR-deficiency in P. aeruginosa and its link to MDR has largely focused on isolates from pwCF or other chronic lung infection contexts. An early study screening intensive care unit isolates attributed the contribution of hypermutation in acute infections of P. aeruginosa to <1%. However, a few recent clinical case studies have suggested MMR-deficient P. aeruginosa may be playing a more prominent role in acute disease contexts. The frequency of MMR-deficiency in different disease contexts was assessed by performing mutational signature analysis on a publicly available dataset of WGS reads from 325 P. aeruginosa clinical isolates from respiratory tract infections (RTIs), urinary tract infections (UTIs), intraabdominal infections (IAIs), and pwCF. Trinucleotide spectra from 22 isolates showed remarkable qualitative similarity with PaΔmutS (FIG. 6A) and were predicted to be MMR-deficient via cosine similarity analysis (FIG. 6B). Predicted MMR-deficient isolates were enriched in pwCF (31%), as expected, but were also in rather appreciable quantity in RTIs (5.5%) and present in UTIs (2.8%) and IAIs (2.7%) (FIG. 6C). MMR-deficient isolates from pwCF are significantly correlated (p=0.0256) with MDR (FIG. 6D) as seen previously. Predicted MMR-deficient isolates trend towards higher rates of MDR in RTIs (FIG. 6E) and UTIs (FIG. 6F), but not IAIs (FIG. 6G). These results suggest a potential larger and more appreciable role of MMR-deficiency in acute infections of P. aeruginosa than previously accepted.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. M. Radman, I. Matic, J. A. Halliday, F. Taddei, Editing DNA replication and recombination by mismatch repair: from bacterial genetics to mechanisms of predisposition to cancer in humans. Philos. Trans. R. Soc. Lond. B. Biol. Sci. 347, 97-103 (1995). doi: 10.1098/rstb.1995.0015.
2. A. Baross-Francis, S. E. Andrew, J. E. Penney, F. R. Jirik, Tumors of DNA mismatch repair-deficient hosts exhibit dramatic increases in genomic instability. Proc. Natl. Acad. Sci. U.S.A. 95, 8739-8743 (1998). doi: 10.1073/pnas.95.15.8739.
3. M. Radman, R. Wagner, Missing mismatch repair. Nature 366, 722 (1993).
4. P. Hsieh, K. Yamane, DNA mismatch repair: Molecular mechanism, cancer, and ageing. Mech. Ageing Dev. 129, 391-407 (2008). doi: 10.1016/j.mad.2008.02.012.
5. H. Lee, E. Popodi, H. Tang, P. L. Foster, Rate and molecular spectrum of spontaneous mutations in the bacterium Escherichia coli as determined by whole-genome sequencing. Proc. Natl. Acad. Sci. U.S.A. 109 (2012), doi:10.1073/pnas.1210309109.
6. R. L. Marvig, H. K. Johansen, S. Molin, L. Jelsbak, Genome Analysis of a Transmissible Lineage of Pseudomonas aeruginosa Reveals Pathoadaptive Mutations and Distinct Evolutionary Paths of Hypermutators. PLoS Genet. 9 (2013), doi:10.1371/journal.pgen.1003741.
7. S. Nik-Zainal, L. B. Alexandrov, D. C. Wedge, P. Van Loo, C. D. Greenman, K. Raine, D. Jones, J. Hinton, J. Marshall, L. A. Stebbings, A. Menzies, S. Martin, K. Leung, L. Chen, C. Leroy, M. Ramakrishna, R. Rance, K. W. Lau, L. J. Mudie, I. Varela, D. J. McBride, G. R. Bignell, S. L. Cooke, A. Shlien, J. Gamble, I. Whitmore, M. Maddison, P. S. Tarpey, H. R. Davies, E. Papaemmanuil, P. J. Stephens, S. McLaren, A. P. Butler, J. W. Teague, G. Jönsson, J. E. Garber, D. Silver, P. Miron, A. Fatima, S. Boyault, A. Langerød, A. Tutt, J. W. M. Martens, S. A. J. R. Aparicio, Å. Borg, A. V. Salomon, G. Thomas, A.-L. Børresen-Dale, A. L. Richardson, M. S. Neuberger, P. A. Futreal, P. J. Campbell, M. R. Stratton, Mutational Processes Molding the Genomes of 21 Breast Cancers. Cell 149, 979-993 (2012). doi: 10.1016/j.cell.2012.04.024.
8. L. B. Alexandrov, S. Nik-Zainal, D. C. Wedge, S. A. J. R. Aparicio, S. Behjati, A. V. Biankin, G. R. Bignell, N. Bolli, A. Borg, A.-L. Børresen-Dale, S. Boyault, B. Burkhardt, A. P. Butler, C. Caldas, H. R. Davies, C. Desmedt, R. Eils, J. E. Eyfjord, J. A. Foekens, M. Greaves, F. Hosoda, B. Hutter, T. Ilicic, S. Imbeaud, M. Imielinski, N. Jager, D. T. W. Jones, D. Jones, S. Knappskog, M. Kool, S. R. Lakhani, C. López-Otin, S. Martin, N. C. Munshi, H. Nakamura, P. A. Northcott, M. Pajic, E. Papaemmanuil, A. Paradiso, J. V. Pearson, X. S. Puente, K. Raine, M. Ramakrishna, A. L. Richardson, J. Richter, P. Rosenstiel, M. Schlesner, T. N. Schumacher, P. N. Span, J. W. Teague, Y. Totoki, A. N. J. Tutt, R. Valdés-Mas, M. M. van Buuren, L. van't Veer, A. Vincent-Salomon, N. Waddell, L. R. Yates, J. Zucman-Rossi, P. Andrew Futreal, U. McDermott, P. Lichter, M. Meyerson, S. M. Grimmond, R. Siebert, E. Campo, T. Shibata, S. M. Pfister, P. J. Campbell, M. R. Stratton, Signatures of mutational processes in human cancer. Nature 500, 415-421 (2013).
9. B. Meier, N. V. Volkova, Y. Hong, P. Schofield, P. J. Campbell, M. Gerstung, A. Gartner, Mutational signatures of DNA mismatch repair deficiency in C. elegans and human cancers. Genome Res. 28, 666-675 (2018). doi: 10.1038/nature12477.
10. M. D. Macia, D. Blanquer, B. Togores, J. Sauleda, J. L. Pérez, A. Oliver, Hypermutation is a key factor in development of multiple-antimicrobial resistance in Pseudomonas aeruginosa strains causing chronic lung infections. Antimicrob. Agents Chemother. 49, 3382-3386 (2005). doi: 10.1128/AAC.49.8.3382-3386.2005.
11. A. M. Smania, I. Segura, R. J. Pezza, C. Becerra, I. Albesa, C. E. Argaraña, Emergence of phenotypic variants upon mismatch repair disruption in Pseudomonas aeruginosa. Microbiology 150, 1327-1338 (2004). doi: 10.1099/mic.0.26751-0.
12. A. Oliver, F. Baquero, J. Blazquez, The mismatch repair system (mutS, mutL and uvrD genes) in Pseudomonas aeruginosa: molecular characterization of naturally occurring mutants. Mol. Microbiol. 43, 1641-1650 (2002). doi: 10.1046/j.1365-2958.2002.02855.x.
13. M. Hogardt, S. Schubert, K. Adler, M. Götzfried, J. Heesemann, Squence variability and functional analysis of MutS of hypermutable Pseudomonas aeruginosa cystic fibrosis isolates. Int. J. of Med. Microbol. 296, 313-320 (2006). doi: 10.1016/j.ijmm.2005.12.018.
14. A. Oliver, R. Cantón, P. Campo, F. Baquero, J. Blázquez, High Frequency of Hypermutable Pseudomonas aeruginosa in Cystic Fibrosis Lung Infection. Science 288, 1251-1253 (2000). doi: 10.1126/science.288.5469.1251.
15. S. Feliziani, A. M. Luján, A. J. Moyano, C. Sola, J. L. Bocco, P. Montanaro, L. F. Canigia, C. E. Argaraña, A. M. Smania, Mucoidy, quorum sensing, mismatch repair and antibiotic resistance in Pseudomonas aeruginosa from cystic fibrosis chronic airways infections. PLoS One. 5, 1-12 (2010). doi: 10.1371/journal.pone.0012669.
16. B. Dößelmann, M. Willmann, M. Steglich, B. Bunk, U. Nübel, S. Peter, R. A. Neher, Rapid and Consistent Evolution of Colistin Resistance in Extensively Drug-Resistant Pseudomonas aeruginosa during Morbidostat Culture. Antimicrob. Agents Chemother. 61 (2017), doi:10.1128/AAC.00043-17.
17. P. P. Khil, A. Dulanto Chiang, J. Ho, J.-H. Youn, J. K. Lemon, J. Gea-Banacloche, K. M. Frank, M. Parta, R. A. Bonomo, J. P. Dekker, Dynamic Emergence of Mismatch Repair Deficiency Facilitates Rapid Evolution of Ceftazidime-Avibactam Resistance in Pseudomonas aeruginosa Acute Infection. mBio 10 (2019), doi:10.1128/mBio.01822-19.
18. Q. Yu, E. F. Griffin, S. Moreau-Marquis, J. D. Schwartzman, B. A. Stanton, G. A. O'Toole, In vitro evaluation of tobramycin and aztreonam versus Pseudomonas aeruginosa biofilms on cystic fibrosis-derived human airway epithelial cells. J. of Antimicrob. Chemother. 67, 2673-2681 (2012). doi: 10.1093/jac/dks296.
19. J. A. Nick, S. M. Moskowitz, J. F. Chmiel, A. v. Forssén, S. H. Kim, M. T. Saavedra, L. Saiman, J. L. Taylor-Cousar, D. P. Nichols, Azithromycin May Antagonize Inhaled Tobramycin When Targeting Pseudomonas aeruginosa in Cystic Fibrosis. Ann. Am. Thorac Soc. 11, 342-350 (2014). doi: 10.1513/AnnalsATS.201310-352OC.
20. E. C. Elson, J. Mermis, D. Polineni, C. M. Oermann, Aztreonam Lysine Inhalation Solution in Cystic Fibrosis. Clin. Med. Insights Circ. Respir. Pulm. Med. 13, 1-10 (2019). doi: 10.1177/1179548419842822.
21. M. Bassetti, A. Vena, A. Croxatto, E. Righi, B. Guery, How to manage Pseudomonas aeruginosa infections. Drugs Context. 7 (2018). doi:10.7573/dic.212527.
22. C. G. Starr, J. Ghimire, S. Guha, J. P. Hoffmann, Y. Wang, L. Sun, B. N. Landreneau, Z. D. Kolansky, I. M. Kilanowski-Doroh, M. C. Sammarco, L. A. Morici, W. C. Wimley, Synthetic molecular evolution of host cell-compatible, antimicrobial peptides effective against drug-resistant, biofilm-forming bacteria. Proc. Natl. Acad. Sci. U.S.A. 117, 8437-8448 (2020). doi: 10.1073/pnas.1918427117.
23. J. Ghimire, R. J. Hart, A. Soldano, C. H. Chen, S. Guha, J. P. Hoffmann, K. M. Hall, L. Sun, B. J. Nelson, T. K. Lu, J. K. Kolls, M. Rivera, L. A. Morici, W. C. Wimley, Optimization of Host Cell-Compatible, Antimicrobial Peptides Effective against Biofilms and Clinical Isolates of Drug-Resistant Bacteria. ACS Infect. Dis. 9, 952-965 (2023). doi: 10.1021/acsinfecdis.2c00640.
24. L. Fernández, R. E. W. Hancock, Adaptive and Mutational Resistance: Role of Porins and Efflux Pumps in Drug Resistance. Clin. Microbiol. Rev. 25, 661-681 (2012). doi: 10.1128/CMR.00043-12.
25. A. Dulanto Chiang, P. P. Patil, L. Beka, J.-H. Youn, A. Launay, R. A. Bonomo, P. P. Khil, J. P. Dekker, Hypermutator strains of Pseudomonas aeruginosa reveal novel pathways of resistance to combinations of cephalosporin antibiotics and beta-lactamase inhibitors. PLoS Biol. 20, e3001878 (2022). doi: 10.1371/journal.pbio.3001878.
26. A. J. McBroom, M. J. Kuehn, Release of outer membrane vesicles by Gram-negative bacteria is a novel envelope stress response. Mol. Microbiol. 63, 545-558 (2007). doi: 10.1111/j.1365-2958.2006.05522.x.
27. Y. Tashiro, N. Nomura, R. Nakao, H. Senpuku, R. Kariyama, H. Kumon, S. Kosono, H. Watanabe, T. Nakajima, H. Uchiyama, Opr86 is essential for viability and is a potential candidate for a protective antigen against biofilm formation by Pseudomonas aeruginosa. J Bacteriol. 190, 3969-3978 (2008). doi: 10.1128/JB.02004-07.
28. N. Jochumsen, R. L. Marvig, S. Damkior, R. L. Jensen, W. Paulander, S. Molin, L. Jelsbak, A. Folkesson, The evolution of antimicrobial peptide resistance in Pseudomonas aeruginosa is shaped by strong epistatic interactions. Nat. Commun. 7 (2016), doi:10.1038/ncomms13002.
29. S. M. Moskowitz, R. K. Ernst, S. I. Miller, PmrAB, a Two-Component Regulatory System of Pseudomonas aeruginosa that Modulates Resistance to Cationic Antimicrobial Peptides and Addition of Aminoarabinose to Lipid A. J Bacteriol. 186, 575-579 (2004). doi: 10.1128/JB.186.2.575-579.2004.
30. D. Owusu-Anim, D. H. Kwon, Differential Role of Two-Component Regulatory Systems (phoQ) and pmrAB) in Polymyxin B Susceptibility of Pseudomonas aeruginosa. Adv. Microbiol. 02, 31-36 (2012). doi: 10.4236/aim.2012.21005.
31. K. P. Romano, T. Warrier, B. E. Poulsen, P. H. Nguyen, A. R. Loftis, A. Saebi, B. L. Pentelute, D. T. Hung, Mutations in pmrB Confer Cross-Resistance between the LptD Inhibitor POL7080 and Colistin in Pseudomonas aeruginosa. Antimicrob. Agents. Chemoth. 63, e00511-19 (2019). doi: 10.1128/aac.00511-19.
32. N. P. Giordano, M. B. Cian, Z. D. Dalebroux, Outer Membrane Lipid Secretion and the Innate Immune Response to Gram-Negative Bacteria. Infec.t Immun. 88, e00920-19 (2020). doi:10.1128/IAI.00920-19.
33. M. V. Douglass, F. Cléon, M. S. Trent, Cardiolipin aids in lipopolysaccharide transport to the gram-negative outer membrane. Proc. Natl. Acad. Sci. U.S.A. 118, e2018329118 (2021). doi:10.1073/pnas.2018329118.
34. P. J. Mogayzel, E. T. Naureckas, K. A. Robinson, C. Brady, M. Guill, T. Lahiri, L. Lubsch, J. Matsui, C. M. Oermann, F. Ratjen, M. Rosenfeld, R. H. Simon, L. Hazle, K. Sabadosa, B. C. Marshall, Cystic Fibrosis Foundation Pulmonary Guideline. Pharmacologic Approaches to Prevention and Eradication of Initial Pseudomonas aeruginosa Infection. Ann. Am. Thorac. Soc. 11, 1640-1650 (2014). doi: 10.1513/AnnalsATS.201404-166OC.
35. P. J. Mogayzel, E. T. Naureckas, K. A. Robinson, G. Mueller, D. Hadjiliadis, J. B. Hoag, L. Lubsch, L. Hazle, K. Sabadosa, B. Marshall, Cystic Fibrosis Pulmonary Guidelines. Am. J Respir. Crit. Care Med. 187, 680-689 (2013). doi: 10.1164/rccm.201207-1160OE.
36. S. E. H. West, B. H. Iglewski, Codon usage in Pseudomonas aeruginosa. Nucleic Acids Res. 16, 9323-9335 (1988).
37. R. J. Grocock, P. M. Sharp, Synonymous codon usage in Pseudomonas aeruginosa PA01. Gene 289, 131-139 (2002).
38. S. T. Parvathy, V. Udayasuriyan, V. Bhadana, Codon usage bias. Mol. Biol. Rep. 49, 539-565 (2022). doi: 10.1007/s11033-021-06749-4.
39. J. D. Cogen, F. Onchiri, J. Emerson, R. L. Gibson, L. R. Hoffman, D. P. Nichols, M. Rosenfeld, Chronic Azithromycin Use in Cystic Fibrosis and Risk of Treatment-Emergent Respiratory Pathogens. Ann. Am. Thorac. Soc. 15, 702-709 (2018). doi: 10.1513/AnnalsATS.201801-012OC.
40. N. Principi, F. Blasi, S. Esposito, Azithromycin use in patients with cystic fibrosis. European J of Clin. Microbiol. & Infect. Dis. 34, 1071-1079 (2015). doi: 10.1007/s10096-015-2347-4.
41. G. Döring, S. P. Conway, H. G. M. Heijerman, M. E. Hodson, N. Hoiby, A. Smyth, D. J. Touw, for the Consensus Committee, Antibiotic therapy against Pseudomonas aeruginosa in cystic fibrosis: a European consensus. European Respiratory Journal 16, 749 (2000). doi: 10.1034/j.1399-3003.2000.16d30.x.
42. S. M. Moskowitz, M. K. Brannon, N. Dasgupta, M. Pier, N. Sgambati, A. K. Miller, S. E. Selgrade, S. I. Miller, M. Denton, S. P. Conway, H. K. Johansen, N. Hoiby, PmrB mutations promote polymyxin resistance of Pseudomonas aeruginosa isolated from colistin-treated cystic fibrosis patients. Antimicrob. Agents Chemother. 56, 1019-30 (2012). doi: 10.1128/AAC.05829-11.
43. Y. Noteboom, D. S. Y. Ong, E. A. Oostdijk, M. J. Schultz, E. de Jonge, I. Purmer, D. Bergmans, J. W. Fijen, J. Kesecioglu, M. J. M. Bonten, Antibiotic-Induced Within-Host Resistance Development of Gram-Negative Bacteria in Patients Receiving Selective Decontamination or Standard Care. Crit. Care Med. 43, 2582-8 (2015). doi: 10.1097/CCM.0000000000001298.
44. R. M. Abd El-Baky, S. M. Masoud, D. S. Mohamed, N. G. Waly, E. A. Shafik, D. A. Mohareb, A. Elkady, M. M. Elbadr, H. F. Hetta, Prevalence and Some Possible Mechanisms of Colistin Resistance Among Multidrug-Resistant and Extensively Drug-Resistant Pseudomonas aeruginosa. Infect. Drug Resist. 13, 323-332 (2020). doi: 10.2147/IDR.S238811.
45. O. Gutiérrez, C. Juan, J. L. Pérez, A. Oliver, Lack of Association between Hypermutation and Antibiotic Resistance Development in Pseudomonas aeruginosa Isolates from Intensive Care Unit Patients. Antimicrob. Agents Chemother. 48, 3573-3575 (2004). doi: 10.1128/AAC.48.9.3573-3575.2004.
46. B. Henrichfreise, I. Wiegand, W. Pfister, B. Wiedemann, Resistance Mechanisms of Multiresistant Pseudomonas aeruginosa Strains from Germany and Correlation with Hypermutation. Antimicrob. Agents Chemother. 51, 4062-4070 (2007). doi: 10.1128/AAC.00148-07.
47. S. Nozick, E. A. Ozer, R. Medernach, R. Kumar, J. O. Mills, R. G. Wunderink, C. Qi, A. R. Hauser, Emergence of a Pseudomonas aeruginosa Hypermutator Strain During the Course of Ventilator-Associated Pneumonia. medRxiv [Preprint](2022). doi:10.1101/2022.05.19.22275029.
48. A. Prunier, B. Malbruny, M. Laurans, J. Brouard, J. Duhamel, R. Leclercq, High Rate of Macrolide Resistance in Staphylococcus aureus Strains from Patients with Cystic Fibrosis Reveals High Proportions of Hypermutable Strains. J Infect. Dis. 187, 1709-1716 (2003). doi: 10.1086/374937.
49. I. Matic, M. Radman, F. Taddei, B. Picard, C. Doit, E. Bingen, E. Denamur, J. Elion, Highly Variable Mutation Rates in Commensal and Pathogenic Escherichia coli. Science 277, 1833-1834 (1997). doi: 10.1126/science.277.5333.1833.
50. P. Komp Lindgren, P. G. Higgins, H. Seifert, O. Cars, Prevalence of hypermutators among clinical Acinetobacter baumannii isolates. J of Antimicrob. Chemother. 71, 661-665 (2016). doi: 10.1093/jac/dkv378.
51. M. A. Jacobs, A. Alwood, I. Thaipisuttikul, D. Spencer, E. Haugen, S. Ernst, O. Will, R. Kaul, C. Raymond, R. Levy, L. Chun-Rong, D. Guenthner, D. Bovee, M. V. Olson, C. Manoil, Comprehensive transposon mutant library of Pseudomonas aeruginosa. Proc. Natl. Acad. Sci. U.S.A. 100, 14339-44 (2003). doi: 10.1073/pnas.2036282100.
52. D. E. Deatherage, J. E. Barrick, Identification of Mutations in Laboratory-Evolved Microbes from Next-Generation Sequencing Data Using breseq. Methods Mol. Biol. 1151, 165-188 (2014). doi: 10.1007/978-1-4939-0554-6_12.
53. H. Wickham, M. Averick, J. Bryan, W. Chang, L. McGowan, R. Frangois, G. Grolemund, A. Hayes, L. Henry, J. Hester, M. Kuhn, T. Pedersen, E. Miller, S. Bache, K. Müller, J. Ooms, D. Robinson, D. Seidel, V. Spinu, K. Takahashi, D. Vaughan, C. Wilke, K. Woo, H. Yutani, Welcome to the Tidyverse. J Open Source Softw. 4, 1686 (2019). doi: 10.21105/joss.01686.
54. J. G. Tate, S. Bamford, H. C. Jubb, Z. Sondka, D. M. Beare, N. Bindal, H. Boutselakis, C. G. Cole, C. Creatore, E. Dawson, P. Fish, B. Harsha, C. Hathaway, S. C. Jupe, C. Y. Kok, K. Noble, L. Ponting, C. C. Ramshaw, C. E. Rye, H. E. Speedy, R. Stefancsik, S. L. Thompson, S. Wang, S. Ward, P. J. Campbell, S. A. Forbes, COSMIC: the Catalogue Of Somatic Mutations In Cancer. Nucleic Acids Res. 47, D941-D947 (2019). doi: 10.1093/nar/gky1015.
55. F. Manders, A. M. Brandsma, J. de Kanter, M. Verheul, R. Oka, M. J. van Roosmalen, B. van der Roest, A. van Hoeck, E. Cuppen, R. van Boxtel, MutationalPatterns: the one stop shop for the analysis of mutational processes. BMC Genomics. 23, 134 (2022). doi: 10.1186/s12864-022-08357-3.

1. A method for treating a hypermutator Pseudomonas infection in a subject in need thereof or preventing hypermutator Pseudomonas-induced antibiotic resistance in a subject having a hypermutator Pseudomonas infection comprising administering to the subject an effective amount of an antipseudomonal small molecule inhibitor and an effective amount of an antipseudomonal cationic peptide.

2. (canceled)

3. The method of claim 1, wherein the antipseudomonal cationic peptide comprises one or more of polymyxin B, D-CONGA, D-CONGA-Q7 or colistin.

4. The method of claim 1, wherein the antipseudomonal small molecule inhibitor comprises one or more of monobactams, fluoroquinolines, cephalosporins, or carbapenems.

5. The method of claim 4, wherein the monobactams comprise one or more of aztreonam, azactam, tigemonam, nocardicin A, tabtoxin, or cayston; or
wherein the fluoroquinolines comprise one or more of ciprofloxacin, gemifloxacin, levofloxacin, delafloxacin, gemifloxacin, moxifloxacin, norfloxacin, or ofloxacin; or
wherein the cephalosporins comprise one or more of: cephalexin, cefadroxil, cephradine, cephalexin, cefazolin, cefuroxime, cefprozil, loracarbef, cefuroxime, cefoxitin, cefotetan, ceftriaxone, cefdinir, cefixime, cefpodoxime, cefditoren, ceftibuten, cefdinir, ceftazidime, cefotaxime, cefoperazone, ceftizoxime, cefepime, ceftaroline, ceftolozane, or cefiderocol; or
wherein the carbapenems comprise one or more of doribax, doripenem, ertapenem, imipenem, cilastatin, relebactam, invanz, meropenem, vaborbactam, merrem IV, primaxin, recarbrio, sulopenem, sulopenem etzadroxil/probenecid, or vabomere.

6. (canceled)

7. (canceled)

8. (canceled)

9. The method of claim 1, wherein the Pseudomonas infection is caused by a Pseudomonas species selected from among P. aeruginosa, P. fluorescens, P. putida, P. cepacia, P. stutzeri, P. maltophilia, and P. putrefaciens.

10. The method of claim 9, wherein the Pseudomonas infection is caused by P. aeruginosa.

11. The method of claim 1, wherein the subject is diagnosed with or is at risk for cystic fibrosis or an acute infection, optionally wherein the acute infection comprises a pressure sore infection, a burn infection, a wound infection, or bloodstream infection.

12. The method of claim 1, wherein the antipseudomonal small molecule inhibitor and the antipseudomonal cationic peptide is administered separately, sequentially, or simultaneously.

13. The method of claim 1, wherein the antipseudomonal small molecule inhibitor or the antipseudomonal cationic peptide is administered orally, intravenously, intramuscularly, intraperitoneally, or subcutaneously.

14. A method for detecting a hypermutator Pseudomonas infection in a subject comprising
sequencing Pseudomonas nucleic acids isolated from a biological sample obtained from the subject;
generating a mutation spectrum of the Pseudomonas nucleic acid sequences; and
detecting the presence of a hypermutator Pseudomonas infection when a Pseudomonas DNA mismatch repair-deficit (dMMR) mutational signature is detected in the mutation spectrum.

15. A method for predicting the risk of antibiotic resistance in a subject having a Pseudomonas infection comprising
sequencing Pseudomonas nucleic acids isolated from a biological sample obtained from the subject;
generating a mutation spectrum of the Pseudomonas nucleic acid sequences; and
determining that the subject is at risk for antibiotic resistance when a Pseudomonas DNA mismatch repair-deficit (dMMR) mutational signature is detected in the mutation spectrum.

16. The method of claim 14, wherein the dMMR mutational signature comprises an increase in C>T transitions in NCC and NCG contexts and/or an increase in T>C transitions in CTN and GTN contexts.

17. The method of claim 14, wherein the biological sample comprises skin tissue, throat swabs, stool, urine, blood, lung tissue, stomach tissue, or urinary tract tissue.

18. The method of claim 14, further comprising administering to the subject an effective amount of an antipseudomonal small molecule inhibitor and an effective amount of an antipseudomonal cationic peptide.

19. A method for preventing multi-drug resistance in a subject infected with Pseudomonas or selecting an antipseudomonal therapy for a subject having a Pseudomonas infection comprising
sequencing Pseudomonas nucleic acids isolated from a biological sample obtained from the subject;
generating a mutation spectrum of the Pseudomonas nucleic acid sequences;
detecting a Pseudomonas DNA mismatch repair-deficit (dMMR) mutational signature in the mutation spectrum; and
administering to the subject an effective amount of an antipseudomonal small molecule inhibitor and an effective amount of an antipseudomonal cationic peptide.

20. (canceled)

21. The method of claim 14, wherein the Pseudomonas infection is caused by a Pseudomonas species selected from among P. aeruginosa, P. fluorescens, P. putida, P. cepacia, P. stutzeri, P. maltophilia, and P. putrefaciens.

22. The method of claim 14, wherein the antipseudomonal cationic peptide comprises one or more of polymyxin B, D-CONGA, D-CONGA-Q7 or colistin.

23. The method of claim 14, wherein the antipseudomonal small molecule inhibitor comprises one or more of monobactams, fluoroquinolines, cephalosporins, or carbapenems.

24. The method of claim 14, wherein Pseudomonas nucleic acids are sequenced via whole genome sequencing (WGS).
